SEQUENCE LISTING (1 sequence)

SEQ ID NO: 1 - protein, 223 aa, severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

(19) United States
(12) Patent Application Publication
(10) Pub. No.: US 2023/0390414 A1
(43) Pub. Date: Dec. 7, 2023

(54) PARTICLE BASED FORMULATION OF SARS-COV-2 RECEPTOR BINDING DOMAIN

(71) Applicant: The Research Foundation for The State University of New York, Amherst, NY (US)

(72) Inventors: Jonathan Lovell; Wei-Chao Huang, Amherst, NY (US)

(21) Appl. No.: 18/247,235

(22) PCT Filed: Sep. 30, 2021
(86) PCT No.: PCT/US21/52907

§ 371 Date: Mar. 29, 2023

Related U.S. Application Data
(60) Provisional application No. 63/085,734, filed on Sep. 30, 2020.

(51) Int. Cl. A61K 47/69; A61K 39/215; A61K 47/54; A61P 31/14

(52) U.S. Cl. CPC A61K 47/6911; A61K 39/215; A61K 47/546; A61P 31/14; A61K 2039/55583

(57) ABSTRACT

Provided are vaccine compositions and methods for generation of immune response (including neutralizing antibodies) against SARS-CoV-2 virus. The vaccine compositions comprise a poly-histidine tagged receptor binding domain (RBD) of the SARS-CoV-2 virus incorporated into a liposome comprising cobalt-porphyrin-phospholipid conjugates, such that one or more histidines of the polyhistidine tag are coordinated to the cobalt of the cobalt-porphyrin and at least a portion of the RBD is exposed to the outside of the liposome.

### CROSS-REFERENCE TO RELATED APPLICATIONS

This application claims priority to U.S. Provisional application No. 63/085,734, filed on Sep. 30, 2020, the disclosure of which in incorporated herein by reference.

### BACKGROUND OF THE DISCLOSURE

SARS-CoV-2 has caused a disruptive worldwide viral pandemic (Petersen et al., The Lancet Infectious Diseases (2020), 20(e238-e244)). The quest for effective vaccine countermeasures is an active pursuit in the biomedical research community (Poland et al., SARS-CoV-2 Vaccine Development: Current Status, Mayo Clinic Proceedings (2020), doi-org/10.1016/j.mayocp.2020.07.021). The spike (S) protein on the virus surface is instrumental for binding, fusing, and entry into host cells, and is also the lead immunogen for several advanced vaccine candidates (Funk et al., Frontiers in Pharmacology 11(937) (2020)). The S protein contains the receptor-binding domain (RBD) that binds to the host receptor, angiotensin-converting enzyme 2 (ACE2). The RBD is an appealing antigen, as most neutralizing antibodies generated during a SARS-CoV-2 infection are directed against it (Nat. Commun. 11(1) 2020); ter Muelen et al., PLoS Med 3(7) (2006) e237). The RBD protein roughly comprises amino acids 330-350 of S.

The RBD has been shown to be a viable immunogen in preclinical studies, conferring protection in non-human primates from viral challenge (Yang et al., Nature (2020), doi.org/10.1038/s441586-020-2599-8n). However, as a relatively small and compact immunogen with 4 internal disulfide bonds, the RBD is expected to exhibit hapten-like properties that limit its immunogenicity, which could necessitate the use of higher antigen doses that would complicate the large scale roll-out of a RBD vaccine. Indeed, it has been shown that immunogenicity is enhanced by engineering the protein construct into dimeric (Dai et al., Cell 182(3) (2020) 722-733.e11) and oligomeric structures (Walls, biorXiv doi.org/10.1101/2020.08.11.247395 (2020)), and another approach necessitated conjugation of the RBD onto a carrier protein (Quinlan et al., 2020, dx.doi.org/10.2139/ssrn.3575134). While effective, such approaches may be time consuming and confound downstream characterization of the RBD.

### SUMMARY OF THE DISCLOSURE

The present disclosure provides compositions and methods for enhancing immunogenicity of SARS-CoV-2 RBD and eliciting specific immune response, including neutralizing antibodies, against SARS-CoV-2.

The present disclosure describes the use of particulate presentation of SARS-CoV-2 RBD to enhance immunogenicity and induce virus neutralizing antibody responses. We demonstrate that when recombinant RBD was admixed with cobalt porphyrin-phospholipid (CoPoP) liposomes, antigen-particles formed rapidly and spontaneously. RBD immunization with CoPoP generated enhanced neutralizing antibodies. Inclusion of an adjuvant, QS21, can enhance the immune response based on providing T cell help. These data indicate that particle presentation of RBD with CoPoP can be used as a safe and potent vaccine against SARS CoV-2.

In an aspect, the present disclosure provides liposomal compositions comprising functionalized nanostructures, wherein the nanostructures have incorporated therein a sequence from the polypeptides/proteins of SARS-CoV-2. An example of protein is the S protein. An example of a sequence from the Spike protein is RBD. The nanostructures may have bilayers. The nanostructures may be in the form of liposomes. The bilayer of the liposomes comprises cobalt porphyrin-phospholipid conjugate, optionally phospholipids that are not conjugated to porphyrin, optionally sterols, and optionally polyethylene glycol (PEG). The liposomes may also comprise one or more adjuvants. One or more sequences of a polypeptide from the SARS-CoV-2 (such as a Spike protein sequence, or the RBD sequence(s) of the Spike protein) having a polyhistidine tag are incorporated into the bilayer such that a portion of the polyhistidine tag resides in the bilayer and at least a portion of the SARS-CoV-2 polypeptide is exposed to the exterior of the bilayer. The liposomes may also have incorporated therein one or more adjuvants.

In an aspect, the present disclosure provides methods for eliciting an immune response against SARS-CoV2. The method comprises administering to a subject in need of immunization, a composition comprising liposomes that comprises cobalt porphyrin-phospholipid conjugate, optionally phospholipids that are not conjugated to porphyrin, optionally sterols, and optionally polyethylene glycol (PEG), and have one or more sequences of a SARS-CoV-2 polypeptide (such as a sequence from the Spike protein, e.g., the RBD sequence) having a polyhistidine tag are incorporated into the bilayer such that a portion of the polyhistidine tag resides in the bilayer and at least a portion of the SARS-CoV-2 polypeptide is exposed to the exterior of the bilayer. The liposomes may also comprise one or more adjuvants. The immune response may comprise humoral response (e.g., generation of antibodies), cellular response (e.g., T cell based response) or both. The antibodies may be neutralizing antibodies. The T-cell response may comprise an increase in CD4+ and/CD8+ cells, e.g., increased numbers of CD4+ or CD8+ cells that secrete IFNγ, IL-2 and TNFα.

In an embodiment, the disclosure provides a vaccine composition comprising

a liposome comprising cobalt porphyrin-phospholipid conjugate, optionally phospholipids that are not conjugated to porphyrin, optionally sterols, and optionally polyethylene glycol (PEG), and have an RBD sequence or a variant thereof having at least a 85% homology therewith, said RBD sequence or variant having a polyhistidine tag incorporated into the bilayer such that at least a portion of the polyhistidine tag resides in the bilayer and at least a portion of the RBD or variant is exposed to the exterior of the liposome. In various embodiments, all or almost all of the RBD or variant thereof is exposed to the exterior of the liposome and all or almost all of the polyhistidine tag resides in the bilayer.

In an embodiment, the disclosure provides a method for immunizing a subject against SARS-CoV-2 infection. The method comprises administering to a subject in need of immunization a composition comprising a plurality of liposomes, at least some of said liposomes comprising cobalt porphyrin-phospholipid conjugate, optionally phospholipids that are not conjugated to porphyrin, optionally sterols, and optionally polyethylene glycol (PEG), and have an RBD sequence or a variant thereof having at least a 85% homology therewith, said RBD sequence or variant having a polyhistidine tag incorporated into the bilayer such that a portion of the polyhistidine tag resides in the bilayer and at least a portion of the RBD or variant is exposed to the exterior of the bilayer. The composition may further comprise one or more adjuvants, which may be incorporated into the liposomes or may be separate from the liposomes within the composition.

In an embodiment, the disclosure provides a method for increasing the immunogenicity of the RBD peptide of SARS-CoV-2 and/or eliciting neutralizing antibodies against SARS-CoV-2 by administering to a subject in need of treatment a composition comprising nanostructures, wherein the nanostructures comprise bilayers, which comprise cobalt porphyrin-phospholipid conjugate, optionally phospholipids that are not conjugated to porphyrin, optionally sterols, and optionally polyethylene glycol (PEG), and have an RBD sequence or a variant thereof having at least a 85% homology therewith, said RBD sequence or variant having a polyhistidine tag incorporated into the bilayer such that a portion of the polyhistidine tag resides in the bilayer and at least a portion of the RBD or variant is exposed to the exterior of the bilayer. Optionally, one or more adjuvants may be incorporated into the nanostructures or administered separately.

### BRIEF DESCRIPTION OF THE FIGURES

FIG. 1. Recombinant RBD binds to CoPoP with intact conformation. A) Ni-NTA bead competition assay. RBD antigen produced in the indicated expression system was incubated with liposomes for 3 hr and then Ni-NTA beads were added and then isolated. Protein that was stably bound to liposomes is in the supernatant (“S”) lanes, whereas unbound proteins is in the bead (“B”) fraction. B) Binding of RBD to CoPoP liposomes after 3 hr incubation as assessed by a high speed centrifugation assay. C) Binding kinetics of a fluorescently labeled RBD to CoPoP liposomes. When the fluorophore-labeled RBD binds CoPoP liposomes, energy transfer results in fluorophore quenching. D) Binding kinetic of the RBD with CoPoP/MPLA or CoPoP/MPLA/QS21 liposomes using RBD produced in HEK293 cells based on Ni-NTA bead competition. E) Slot blot detection of ACE2 binding to adsorbed RBD or Pfs25 (an unrelated control antigen) in soluble or particulate form. F) Binding of fluorophore-labeled RBD, in soluble or particulate form, to hACE2-expressing cells. G) Binding of liposomes themselves (based on PoP signal) decorated with the RBD or the unrelated Pfs25 control antigen to hACE2 coated plate.

FIG. 2. CoPoP/RBD particles are small, stable, and preferentially taken up by immune cells. A) Size of liposomes following RBD binding, measured by dynamic light scattering. For each set, from left to right, the bars are: no antigen, RBD (HEK293), and RBD (sf9). B) Cryo electron micrographs of RBD (HEK293) bound to indicated liposomes. A 100 nm scale bar is shown. C) Particle stability with a week-long incubation in 20% human serum incubated at 37° C., as measured by the association of a fluorophore labeled RBD to the liposomes. D) RBD uptake in vitro following incubation with murine RAW264.7 or bone marrow-derived dendritic cells (BMDC) cells. Cytochalasin B was used as a phagocytosis inhibitor and chlorpromazine was used as an endocytosis inhibitor. E) RBD uptake in immune cells within draining lymph nodes in vivo following intramuscular immunization of mice. Labeled RBD uptake was assessed with flow cytometry and co-staining with the indicated surface markers. Bar graphs in A and D show mean +/−std. dev. for n=3 measurements. For E, data were analyzed by one-way ANOVA followed by Tukey's post hoc analysis adjusting for multiple comparisons, p*<0.05, p**<0.01.

FIG. 3. Functional assessment of mouse antibodies induced by the RBD admixed with various vaccine adjuvants. Outbred mice were immunized with 100 ng RBD admixed with indicated adjuvant on day 0 and day 14 prior to serum collection on day 28. A) Anti-RBD IgG titer. B) Pseudovirus IC₅₀ inhibition titer. C) Inhibition in a surrogate virus neutralization test that measures interaction between the RBD and hACE2. D) Live SARS-CoV-2 virus neutralizing titers in post immune mouse sera. For A and B, log10 transformed titer were analyzed by one-way ANOVA test followed by Tukey's comparisons, there is no statistical different between CoPoP liposomes with or without QS21; other adjuvants all show significant difference with p<0.005 when compared to CoPoP liposomes. For C and D, data were analyzed by one-way ANOVA followed by Tukey's comparisons, there is no statistical different between CoPoP liposomes with or without QS21; other adjuvants all show significant difference with p<0.001 when compared to CoPoP liposomes.

FIG. 4. Rabbit RBD immunization. Rabbits were immunized with 20 μg RBD admixed with the indicated adjuvants on day 0 and 21, and day 42 serum was collected. Anti-RBD IgG titer A) and pseudovirus neutralization B) at indicated time points C). Inhibition in a surrogate virus neutralization test that measures interaction between the RBD hACE2. D) Live SARS-CoV-2 virus neutralization using post-immune sera. For A and B, logio transformed titer were analyzed by one-way ANOVA followed by Tukey's comparisons. For C and D, data were analyzed by one-way ANOVA followed by Tukey's comparisons. p*<0.05, p**<0.01, p***<0.005, p****<0.001.

FIG. 5. Antibody and cellular immune activation. A) Recruitment of immune cells in draining lymph nodes of mice, 48 hours after intramuscular administration. For each set, from left to right, the bars are: Control, CoPoP/MPLA/QS21, CoPoP/MPLA, and Alum. Generation of germinal center activation were identified using flow cytometry, population of B) Germinal center B cells and C) Tfh cells were measured from lymph nodes after 1-week post immunization with 100 ng of RBD (HEK293) admixed with CoPoP liposomes or Alum. Splenocytes were collected from immunized mice and stimulated with RBD antigen. D) IFN-γ secretion. E) Intracellular staining triple cytokines (IFNγ, IL-2 and TNFα) in CD4+ T cells. For A-E, data were analyzed by one-way ANOVA test followed by Tukey's comparisons. p*<0.05, p**<0.01, p***<0.005, p****<0.001). For A and E, the line in the box represent the median and the whiskers issuing from the box extend to the group minimum and maximum value. The length of the box represents the interquartile range.

FIG. 6. RBD immunization with CoPoP is well-tolerated. A) local reactogenicity of CoPoP liposomes compared to other adjuvants (all mixed prior to injection). B) Weight change of mice after immunization with 1 μg RBD (ten-fold higher than functional dose) with n=5 mice per group. C) Cobalt level in serum following 100 ng RBD immunization prime and boost with the indicated adjuvants. Bar graphs in A show mean +/−std. dev. for n=4 mice/group. For A, data were analyzed by one-way ANOVA test followed by Tukey's comparisons. p*<0.05, p**<0.01, p****<0.001).

FIG. 7. Schematic representation of binding assay and conformation assay of His-tagged RBD with CoPoP liposomes.

FIG. 8. RBD on CoPoP liposome surface recognize ACE2 on Slot Blot. A) Different doses of free RBD or RBD with CoPoP/MPLA liposome on membrane. Pfs25 was used as a negative control. B) Comparison between RBD with CoPoP/MPLA liposomes and RBD with CoPoP/MPLA/QS21 liposomes.

FIG. 9. RBD on CoPoP liposome surface was recognized by specific antibodies on Slot Blot.

FIG. 10. RBD_DY490 binds to ACE2/HEK293.

FIG. 11. Liposomal distribution was measured using DLS. A) CoPoP/MPLA and B) CoPoP/MPLA/QS21 liposomes. Liposomes were incubated with RBD(HEK293) or RBD(sf9) for 3 hr at room temperature.

FIG. 12. Gating strategy for APC uptake of RBD in lymph nodes. Cells were gated by SSC-FSC then CD11c-APC, F4/80-PE, B220-APC and IA-IE-positive antigen uptake was assessed by using DY490 labeled RBD. Representative plots are shown from biologically independent experiments with n=5 mice.

FIG. 13. His-tagged RBD with CoPoP liposomes is immunogenic in outbreed mice. Mice were immunized with A) RBD-HEK293 and B) RBD-sf9 at the time points indicated by arrows.

FIG. 14. Pseudovirus entry in ACE2/HEK293 and HEK293 cells.

FIG. 15. Sera dilution and percentage of inhibition of Psv entry into ACE-HEK293 cells. Sera from mice immunized with A) RBD-HEK293 antigen or B) RBD-sf9 admixed with indicated adjuvant.

FIG. 16. Binding ability of His-tagged RBD to CoPoP liposomes for rabbit immunization.

FIG. 17. Rabbits were immunized with 20 μg of RBD with CoPoP liposomes or Alum on Day 0 and Day 42. A) Immunized Sera were incubated with Psv at different dilution factors. A dose dependent inhibition was observed from the final bleeding sera from rabbit immunized with CoPoP liposomes but not the Alum group. B) Weight of rabbit post-immunization.

FIG. 18. Recruitment of immune cells in the draining lymph node. A) Dot plot of lymph node cells collected 48 hr after CoPoP/MPLA liposomes injection. x-y axis refers to CD11c-APC and CD11b-PE cy7. B) Region 1 includes macrophages, infiltrating monocytes, neutrophils and eosinophils. C) Region 2 represents mDC, region 3 represents CD11b^{low} DC and region 4 represents CD11b⁻DC.

FIG. 19. Gating strategy for germinal center (GC) activation. GC cells (GL7⁺CD95⁺; within the B220⁺ cell population) were gated with A) B220 surface marker to identified B cells, followed by B) gating GL7⁺CD95⁺ population. Tfh cells (CXCR5⁺PD-1⁺; within the CD4 ⁺ cell population), cells were gated with C) CD4 surface marker to identify CD4⁺ T cells, followed by D) gating CXCR5⁺PD-1⁺.

FIG. 20. Isotype ratios of CoPoP liposomes, ISA720 and PoP liposomes. A) IgG1 and B) IgG2a.

FIG. 21. Gating of CD4⁺ T cells and CD8⁺ T cells. A) Live and Dead cells were gated, then TCRbeta⁺CD4⁺ T cells were gated, followed by gating CD44^{high} population. Later on Foxp- population were gated, and IL2⁺ cells were gated, followed by gating B) TNFα⁺IFNγ⁺ cell population. C) Live and Dead cells were gated, then TCRbeta⁺CD8⁺ T cells were gated, followed by gating CD44^{high} population. Later on IL2⁺ cells were gated, followed by gating D) TNFα⁺IFNγ⁺ cell population.

FIG. 22. Splenocytes were collected from immunized mice, and stimulated with RBD antigen. Intracellular staining of signal cytokine in A) CD4⁺ T cells and B) CD8⁺ T cells. Triple cytokines in C) CD8⁺ T cells. From left to right for each set, the bars are: CoPoP/MPLA/QS221, CoPoP/MPLA, PoP/MPLA, AS01-like, Alum, ISA720, Addavax, and Control.

FIG. 23. Tolerability of CoPoP liposomes with 1 μg His-tagged RBD in CD-1 mice. Mice were treated with “CoPoP/MPLA+RBD” (1 μg Pfs25, 4 ng CoPoP and 1.6 μg MPLA) or “CoPoP/MPLA/QS21+RBD” (1 μg Pfs25, 4 μg CoPoP and 1.6 μg MPLA and 1.6 μg QS21). Values show mean +/−std. dev for n=6 mice per group.). A) Complete blood count parameters are as follows for red blood cells: RBC (red blood cell count), HGB (hemoglobin), HCT (hematocrit); MCV (mean cell volume); MCH (mean cell hemoglobin), MCHC (mean cell hemoglobin concentration) and RDW (red cell distribution width); white blood cell parameters are as follows: WBC (white blood cells), NEU (neutrophils), LYM (lymphocytes), MONO (monocytes); EOS (Eosonophils), BAS (Basophil). platelets parameters are as follows: PLT (platelet) and MPV (mean platelet volume). B) Serum markers with their general description are as follows. Kidney function markers are as follows: BUN (blood urea nitrogen), CREA (creatinine), PHOS (phosphorus), Ca⁺ (calcium), pancreas function is as follows: Protein TP (total protein), ALB (albumin), GLOB (globulin) other GLU (glucose), CHOL (cholesterol). liver function are as follows: ALT (alanine aminotransferase), ALP (alkaline phosphatase), ALB (albumin), TBIL (total bilirubin). The line in the box represent the median and the whiskers issuing from the box extend to the group minimum and maximum value. The length of the box represents the interquartile range. Unpaired two-sided student's T-test were used for statistical analysis, * represent the comparison between CoPoP liposomes to control with p<0.1 and ** represent the comparison between CoPoP liposomes to control with p<0.05. From left to right for each set, the bars are: CP, CPQ, and Untreated.

FIG. 24. Table 1. Comparison of Complete blood count (CBC) parameters between untreated mice and mice treated with CoPoP liposome in CD-1 mice. Mice were treated with “CoPoP/MPLA+RBD” (1 μg RBD, 4 μg CoPoP and 1.6 μg MPLA) or “CoPoP/MPLA/QS21+RBD” (1 μg Pfs25, 4 μg CoPoP and 1.6 μg MPLA and 1.6 μg QS21). Values show mean +/−std. dev for n=6 mice per group. CBC parameters are as follows for red blood cells: RBC (red blood cell count), HGB (hemoglobin), HCT (hematocrit); MCV (mean cell volume); MCH (mean cell hemoglobin), MCHC (mean cell hemoglobin concentration) and RDW (red cell distribution width); white blood cell parameters are as follows: WBC (white blood cells), NEU (neutrophils), LYM (lymphocytes), MONO (monocytes); EOS (Eosonophils), BAS (Basophil). Platelets parameters are as follows: PLT (platelet) and MPV (mean platelet volume).

FIG. 25. Table 2. Comparison of Blood Chemistry Panel between untreated or CoPoP liposome-treated CD-1 mice. Mice were treated with “CoPoP/MPLA+RBD” (1 μg RBD, 4 μg CoPoP and 1.6 μg MPLA). Values show mean +/−std. dev for n=6 mice per group. Serum markers with their general description are as follows. Kidney function markers are as follows: BUN (blood urea nitrogen), CREA (creatinine), PHOS (phosphorus), Ca⁺ (calcium), pancreas function is as follows: Protein TP (total protein), ALB (albumin), GLOB (globulin) other GLU (glucose), CHOL (cholesterol). liver function are as follows: ALT (alanine aminotransferase), ALP (alkaline phosphatase), ALB (albumin), TBIL (total bilirubin).

FIG. 26. Table 3 Summary of murine immunization data

FIG. 27. Table 4: Summary of rabbit immunization data.

### DESCRIPTION OF THE DISCLOSURE

Throughout this application, the use of the singular form encompasses the plural form and vice versa. For example, “a”, or “an” also includes a plurality of the referenced items, unless otherwise indicated.

Every numerical range given throughout this specification includes its upper and lower values, as well as every narrower numerical range that falls within it, as if such narrower numerical ranges were all expressly written herein, and every value is included to the tenth of the value of the lower limit.

The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

The term “effective amount” as used herein refers to an amount of an agent

sufficient to achieve, in a single or multiple doses, the intended purpose of treatment or administration. The exact amount desired or required will vary depending on the particular compound or composition used, its mode of administration, patient specifics and the like. Appropriate effective amount can be determined by one of ordinary skill in the art informed by the instant disclosure using only routine experimentation.

The receptor-binding domain (RBD) of the SARS-CoV-2 spike protein is a candidate vaccine antigen that binds human Angiotensin-converting enzyme 2 (ACE2), leading to virus entry. Here, we show that rapid conversion of the recombinant RBD into particulate form via admixing with liposomes containing cobalt-porphyrin-phospholipid (CoPoP) potently enhances functional antibody response. The present compositions may be used for generation of humoral (e.g., generation of antibodies) and/or cellular (T-cell mediated) immune response. The T-cell response may involve CD4+, CD8+ and/or other immune cells. Administration of the present compositions may result in an increase in adaptive response and/or innate response against the SARS-CoV-2 virus. Data provided here demonstrate that administration of the present compositions elicited SARS-CoV-2 neutralizing antibodies. The present compositions were able to elicit both cellular and humoral response. The cellular response may be in the form of increased numbers or activity of T cells. For example, increased cytokine producing cells (CD4+ and CD8+) were observed for IFNγ, IL-2 and TNFα.

Data provided here demonstrates that antigen binding via His-tag insertion into the CoPoP bilayer results a serum-stable and conformationally-intact display of RBD on the liposome surface. Compared to other vaccine formulations, it was surprising to find that immunization using CoPoP liposomes admixed the RBD induces multiple orders of magnitude higher levels of antibody titers in mice that neutralize pseudovirus cell entry, block RBD interaction with ACE-2, and/or inhibit live virus replication in human cells. Enhanced immunogenicity may be accounted for by greater RBD uptake into antigen presenting cells in particulate form, and also improved immune cell infiltration in draining lymph nodes. When adjuvant QS21 is used, it may account for enhanced antigen-specific polyfunctional T cell response. In an animal model, high dose immunization resulted in minimal local reactogenicity was well-tolerated, and did not elevate serum cobalt levels. Taken together, these results show that particulate presentation of RBD immunogen can be used for inducing neutralizing antibody responses against SARS-CoV-2.

The term “neutralizing” as used herein in reference to an antibody (e.g., antibody generated as part of a host immune response) refers to antibody or the antigen binding fragment that inhibits SARS-CoV-2 virus from infecting a target cell for replication, regardless of the mechanism by which neutralization may be achieved. For example, the virus may be neutralized by inhibiting the entry of SARS-CoV-2 into host mammalian cells or inhibiting entry of pseudotype viruses displaying the Spike protein of SARS-CoV-2 into host mammalian cells. The term “pseudovirus” refers to recombinant viral particles containing a reporter gene that also expresses the Spike protein of SARS-CoV-2 on its surface.

In an aspect, this disclosure provides a method for preventing or reducing the severity of SARS-CoV-2 infection in a subject (e.g., a human subject) comprising administration to a subject in need thereof, a composition comprising liposomes, wherein the bilayer of the liposomes comprise cobalt-porphyrin-phospholipid such that the cobalt resides within the bilayer, and is coordinated to one or more histidines of a polyhistidine tagged SARS-CoV-2 protein or peptide (e.g., Spike protein, e.g., a receptor binding domain of the Spike protein, e.g., having a sequence of SEQ ID NO:1 or a variant thereof having at least a 90% homology thereto). The administration of the compositions can be used for eliciting specific humoral and/or cellular responses against SARS-CoV-2 infection. The humoral and/or the cellular immune responses are greater than responses elicited with administration of the RBD when the RBD is not incorporated into the present CoPoP liposomes.

In an aspect, the disclosure provides compositions comprising nanostructures, wherein the nanostructures (e.g., in the form of liposomes) comprise a bilayer comprising porphyrin-phospholipid conjugates that have cobalt chelated thereto such that the cobalt resides within the bilayer, and one or more peptide molecules having a sequence of SEQ ID NO:1 or a variant thereof having at least a 85% or at least a 90% homology thereto and having poly-histidine tag such that the poly-his tag can coordinate with the cobalt residing within the bilayer and at least a portion of the RBD molecule is exposed on the surface of the nanostructure. The bilayers can optionally have phospholipids that are not conjugated to a porphyrin, optionally have sterols, and optionally have polyethylene glycol (PEG). The liposome may further comprise one or more adjuvants. Examples of adjuvants include MF59, QS21, and attenuated lipid A derivatives such as monophosphoryl lipid A, or synthetic derivatives such as 3-deacylated monophosphoryl lipid A, or Monophosphoryl Hexa-acyl Lipid A, 3-Deacyl. Combinations of adjuvants may also be used. Monophosphoryl lipid A has a lipid-like structure, and therefore it is also present in the lipid bilayer while forming the liposomes.

In an aspect, the disclosure provides a vaccine composition comprising nanostructures (e.g., in the form of liposomes) comprising a bilayer comprising porphyrin-phospholipid conjugates, optionally phospholipids that are not conjugated to a porphyrin, optionally sterols, and optionally polyethylene glycol (PEG), where the porphyrin-phospholipid conjugates have cobalt chelated thereto such that the cobalt resides within the bilayer, and one more peptide molecules having a sequence of SEQ ID NO:1 or a variant thereof having at least a 85% homology thereto and having poly-histidine tag such that the poly-his tag can coordinate with the cobalt residing within the bilayer and at least a portion of the RBD molecule is exposed on the surface of the nanostructures, and optionally an adjuvant, wherein the adjuvant may be incorporated into the liposomes or may be present in the composition without being incorporated into the liposomes, or both.

In an embodiment, the present disclosure provide a vaccine composition effective against the SARS-CoV-2 virus infection comprising i) a polyhistidine-tagged amino acid sequence of receptor-binding-domain (RBD) of Spike protein from SARS-CoV-2, ii) an adjuvant, wherein the adjuvant comprises liposomes comprising i) a bilayer, wherein the bilayer comprises one or more phospholipids, and one or more porphyrins having cobalt coordinated (e.g., chelated) thereto forming cobalt-porphyrin, and the cobalt-porphyrin is conjugated to at least some of the phospholipids forming cobalt-porphyrin-phospholipids; and wherein the RBD is incorporated into the liposome such that a portion of the polyhistidine tag resides in the hydrophobic portion of the bilayer and one or more histidines of the polyhistidine tag are coordinated to the cobalt in the cobalt-porphyrin and a portion of the RBD is exposed to the exterior of the liposome, and iii) a pharmaceutical carrier. The vaccine composition may comprise additional adjuvants, e.g., QS21.

The terms nanostructures and liposomes are used interchangeably in this disclosure.

In an aspect, the present disclosure provides nanostructures comprising at least a bilayer. The bilayer comprises porphyrin-phospholipid conjugates that have cobalt chelated thereto such that the cobalt resides within the bilayer. The bilayer structures can form liposomes. The structures can comprise two monolayers (bilayers), where the hydrophobic groups of the two monolayers are opposed and the hydrophilic groups are exposed to the exterior. The disclosure herein regarding bilayers is also applicable to monolayers. The bilayers or monolayers are sometimes referred to herein as “membranes”.

The bilayers of the nanostructures of the present disclosure have one or more SARS-CoV-2 polypeptides or portions thereof, incorporated therein. For example, the present liposomes may have portions of his-tagged Spike protein of the SARS-CoV-2 incorporated therein. An example of a portion of the Spike protein is the RBD region. In an embodiment, the liposomes of the present disclosure have a sequence or sequences of the RBD incorporated therein. The liposomes may additionally have one or more adjuvants incorporated therein.

Some or all of the cobalt porphyrins in the bilayer can non-covalently bind polyhistidine-tagged molecules (such as a RBD sequence of SARS-CoV-2), such that at least part of the polyhistidine tag resides within the bilayer and the tagged molecule is presented on the surface of the bilayer. In the present bilayers, it is considered that one or more histidine residues in the polyhistidine tag are coordinated to the cobalt within the bilayer, thereby providing stability to the structure. The entire histidine tag may reside within the bilayer. A porphyrin phospholipid conjugate having cobalt conjugated thereto is referred to herein as CoPoP. Liposomes wherein the bilayer comprises CoPoP are referred to herein as CoPoP liposomes. A porphyrin phospholipid conjugate without cobalt conjugated thereto is referred to herein as PoP. Liposomes wherein the bilayer comprises PoP only (i.e., no CoPoP) are referred to herein as PoP liposomes. CoPoP liposomes may also comprise PoP. The CoPoP liposomes can be functionalized with histidine tagged molecules. The term “his-tagged molecules” as used herein means molecules—such as, for example, peptides, polypeptides, or proteins—which have a histidine tail. For example a peptide with a histidine tail is a his-tagged molecule. Such his-tag containing CoPoP liposomes are referred to herein as his-tagged CoPoP liposomes or his-tagged CoPoP.

As used herein, “phospholipid” is a lipid having a hydrophilic head group having a phosphate group connected via a glycerol backbone to a hydrophobic lipid tail. The phospholipid comprises an acyl side chain of 6 to 22 carbons, including all integer number of carbons and ranges therebetween. In certain embodiments, the phospholipid in the porphyrin conjugate is 1-palmitoyl-2-hydroxy-sn-glycero-3-phosphocholine. The phospholipid of the porphyrin conjugate may comprise, or consist essentially of phosphatidylcholine (PC), phosphatidylethanoloamine (PE), phosphatidylserine (PS) and/or phosphatidylinositol (PI). Examples of phospholipids include, but are not limited to, Dipalmitoylphosphatidylcholine (DPPC), Dioleoyl phosphatidylcholine (DOPC), Dimyristoylphosphatidylcholine (DMPC), Distearoylphosphatidylcholine (DSPC), Distearoyl phosphatidylethanolamine (DSPE) and the like.

In an embodiment, the present disclosure provides liposomes wherein the bilayer comprises CoPoP, and one or more his-tagged RBD sequences, wherein at least part of the polyhistidine tag resides within the bilayer and at least part of the RBD is presented on the surface of the bilayer. The liposome may further comprise one or more adjuvants. Examples of adjuvants include attenuated lipid A derivatives such as monophosphoryl lipid A (MPLA), or synthetic derivatives such as3-deacylated monophosphoryl lipid A, or Monophosphoryl Hexa-acyl Lipid A, 3-Deacyl.

An adjuvant can be used as a 0.001 to 50 wt % solution in phosphate buffered saline, and the antigen is present in the order of micrograms to milligrams, such as about to about 5 wt %, such as about 0.0001 to about 1 wt %, or such as about 0.0001 to about 0.05 wt %. The antigen can be present in an amount in the order of micrograms to milligrams, or, about 0.001 to about 20 wt %, such as about 0.01 to about 10 wt %, or about to about 5 wt %.

The adjuvant can be administered as a separate component in the immunogenic compositions or it can be incorporated into the liposome. Examples of adjuvants include complete Freund's adjuvant, incomplete Freund's adjuvant, monophosphoryl lipid A (MPLA), aluminum phosphate, aluminum hydroxide, alum, phosphorylated hexaacyl disaccharide (PHAD), Sigma adjuvant system (SAS), AddaVax (Invitrogen), MF59, QS21, saponin, and combinations thereof. Other carriers like wetting agents, emulsifiers, fillers, and the like may also be used. Synthetic MPLA can be used in the present compositions and methods, including PHAD, PHAD-504, and 3D6A-PHAD. In embodiments, liposomes may be formed with 4:2:1:X DPPC:Cholesterol:CoPoP:MPLA, where MPLA was each of these types of synthetic versions, and Xis 5, 4, 3, 2, or 1.

In an embodiment, the CoPoP/MPLA liposome formulation can have a mass ratio of [DPPC:CHOL:MPLA:CoPoP] [4:2:0.4:1]. In embodiments where QS21 is also present, the CoPoP/MPLA/QS21 liposome formulation may have a mass ratio of [DOPC:CHOL:MPLA:CoPoP:QS21] [20:5:0.4:1:0.4]. AS01-like liposome formation may have a mass ratio of [DOPC:CHOL:MPLA:QS21] [20:5:0.4:0.4] The ranges for the CoPoP/MPLA liposome formulation could be from [DOPC:CHOL:MPLA:CoPoP:QS21] [20:5:0.1-1:1:0.1-1] by changing the mass ratio of MPLA and QS21. The range of his-tagged polypeptide to liposomes can be from 1:1 to 1:8 mass ratio of his-tagged polypeptide peptide to CoPoP.

The sequence of RBD is as follows, excluding a secretion signal peptide and a C-terminus his-tag.

### RBD Sequence:

(SEQ ID NO: 1)
RVQPTESIVRFPNITNLCPFGEVFNATRFASVYAWNR
KRISNCVADYSVLYNSASFSTFKCYGVSPTKLNDLCF
TNVYADSFVIRGDEVRQIAPGQTGKIADYNYKLPDDF
TGCVIAWNSNNLDSKVGGNYNYLYRLFRKSNLKPFER
DISTEIYQAGSTPCNGVEGENCYFPLQSYGFQPTNGV
GYQPYRVVVLSFELLHAPATVCGPKKSTNLVKNKCVN
F

In embodiments, variants of the above sequence whose sequence may be at least 90%, 95%, 96%, 97%, 98% or 99% identical to SEQ ID NO:1 may be used. Longer sequences comprising the sequence of SEQ ID NO:1 or its variants may be used. Longer sequences may comprise one or more signal peptide sequence, secretion sequence, C-terminus his-tag, cloning related sequences and the like, or any other amino acids. In embodiments, shorter portions of SEQ ID NO:1 may be used.

The RBD or other sequences may contain substitutions to modify the immunogenicity. The modified sequences may have only naturally occurring amino acids, or may be a mixture of naturally occurring and non-naturally occurring amino acids. In some embodiments, RBD sequences or variants thereof may be the only amino acid sequences present in the liposomes.

Nanostructures containing his-tagged CoPoP bilayers, which have RBD sequences attached to the histidine tag exhibit desirable stability. The his-tagged RBD sequences are non-covalently attached to (coordinated to) the CoPoP and can be prepared by an incubation process. Therefore, the process does not need removal or passivation of reactive moieties—such as maleimide and the like—or exogenous catalysts or non-natural amino acids that are used in other types of conjugation chemistries.

The cobalt porphyrin-phospholipid can make up from 1 to 100 mol % of the bilayer, including 0.1 mol % values and ranges therebetween. For example, the cobalt porphyrin-phospholipid can make up from 1 to 20 mole %, or from 5 to 10 mol % of the bilayer. If the cobalt porphyrin-phospholipid makes up 100% of the bilayer, then there are no phospholipids present that are not conjugated to cobalt porphyrin. The bilayer can also comprise one or more sterols and/or polyethylene glycol. The sterol can be cholesterol. In an embodiment, the CoPoP is present in the nanoparticles from 0.1 to 10 mol % with the remainder 99.9 to 90 mol % being made up by additional lipids (the percent being of the entire bilayer lipids). For example, the combination of CoPoP and PoP can be present from to 10 mol %, sterol can be present from 0.1 to 50 mol %, optionally, attenuated lipid A derivatives such as monophosphoryl lipid A or 3-deacylated monophosphoryl lipid A or a related analog can be present from 0 to 20 mol % or 0.1 to 20 mol %, and the remainder can be made up by additional phospholipids. In an embodiment, the combination of CoPoP and PoP may be present in the nanoparticles from 0.1 to 10 mol % with the remaining 99.9 to 90 mol % being made up by additional phospholipids. For example, the combination of CoPoP and PoP can be present from 0.1 to 10 mol %, sterol can be present from 0 to 50 mol % or 0.1 to 50 mol %, optionally PEG can be present from 0 to 20 mol % or 0.1 to 20 mol %, and the remainder can be made up by phospholipids, such as DOPC, DSPC, DMPC or combinations thereof and the sterol, if present, can be cholesterol.

The number of histidines in the polyhistidine-tag in the bilayer can be from 2 to 20. For example, the number of histidines in the polyhistidine-tag can be from 6 to 10. For example, the number of histidines can be 2, 3, 4, 5, 6, 7, 8, 9, 10, 11, 12, 13, 14, 15, 16, 17, 18, 19, or 20. The histidine tag (his-tag) may carry a variety of presentation molecules of interest for various applications. At least one or both ends of the his-tag can reside close to the outer surface of the liposome. In embodiments, at least one end of the polyhistidine tag is covalently attached to a presentation molecule. In an embodiment, one end (e.g., the C-terminus) of the his-tag is free (e.g., if the end is the C-terminus, the terminus is an unprotected amide or unprotected carboxylic acid) and a peptide (such as the RBD sequence) is attached to the other end (e.g., the N-terminus). It is considered that at least a part of the his-tag is located within the bilayer such that it is coordinated to cobalt contained therein.

The liposomes may be spherical or non-spherical. The size of the liposomes can be from 50 to 1000 nm or more. In an embodiment, the liposomes have a size (e.g., a longest dimension such as, for example, a diameter) of 50 to 1000 nm, including all integer nm values and ranges therebetween. For example, the size may be from 50 to 200 nm or from to 1000 nm. If the liposomes are not spherical, the longest dimension can be from 50 to 1000 nm. These dimensions can be achieved while preserving the nanostructure width of the bilayer. The RBD sequence or portion thereof can be incorporated in the bilayer. The liposomes can additionally carry cargo in the aqueous compartment. In an embodiment, the liposomes can have a size of 30 nm to 250 nm, including all integers to the nm and ranges therebetween. In an embodiment, the size of the liposomes is from 100-175 nm. In an embodiment, at least 50%, 60%, 70%, 80%, 85%, 90%, 95%, 99%, or 100% of the liposomes in the composition have a size of from 30 to 250 nm or from 100 to 175 nm. The liposomes or nanostructures can be more than 200 nm. In an embodiment, the nanostructures are more than 1000 nm. In an embodiment, the nanostructures are from 200 to 1000 nm. In an embodiment, the largest dimensions of the nanostructure are less than 200 nm, while preserving the nanostructure width of the bilayer. In an embodiment, the size of the nanostructures exceed 200 nm in some dimensions, while preserving the nanostructure width of the bilayer. In an embodiment, the size of the nanostructures exceed 1000 nm in some dimensions, while preserving the nanostructure width of the bilayer.

The liposomes, or nanoparticles having a coating or bilayer, as described herein can have additional presentation molecules presented thereon, which can be antigenic molecules and/or targeting molecules. The presentation molecules can also provide targeting ability and/or imaging or other functionalities.

Liposomes or other nanostructures comprising his-tagged polypeptides and CoPoP compositions exhibit high serum-stability with respect to binding of the his-tagged polypeptide to the liposome. In an embodiment, when incubated with serum (such as diluted serum) at room temperature, more than 60% of the his-tagged peptide remains bound to the CoPoP-containing bilayer after 24 hours incubation. In an embodiment, more than 85% of the his-tagged peptide remains bound to the CoPoP layer after incubation with serum (such as 50% serum or more) for 24 hours. Thus, these structures can be stable under serum or concentrated or diluted serum conditions.

In the present structures, the cobalt-porphyrin of the bilayers is a porphyrin having a cobalt (Co) cation conjugated to the porphyrin. The porphyrin can be conjugated to a phospholipid (referred to herein as a cobalt porphyrin-phospholipid or cobalt porphyrin-phospholipid conjugate). The porphyrin portion of the cobalt-porphyrin or cobalt-porphyrin conjugate making at least part of some of the bilayer of the liposomes or other structures comprises porphyrins, porphyrin derivatives, porphyrin analogs, or combinations thereof. Exemplary porphyrins include hematoporphyrin, protoporphyrin, and tetraphenylporphyrin. Exemplary porphyrin derivatives include pyropheophorbides, bacteriochlorophylls, Chlorophyll A, benzoporphyrin derivatives, tetrahydroxyphenyl chlorins, purpurins, benzochlorins, naphthochlorins, verdins, rhodins, keto chlorins, azachlorins, bacteriochlorins, tolyporphyrins, and benzobacteriochlorins. Exemplary porphyrin analogs include expanded porphyrin family members (such as texaphyrins, sapphyrins and hexaphyrins) and porphyrin isomers (such as porphycenes, inverted porphyrins, phthalocyanines, and naphthalocyanines). For example, the cobalt-porphyrin can be a vitamin B12 (cobalamin) or derivative. In an embodiment, the CoPoP is cobalt-pyropheophorbide-phospholipid. The structure of pyropheophorbide-phospholipid is shown below:

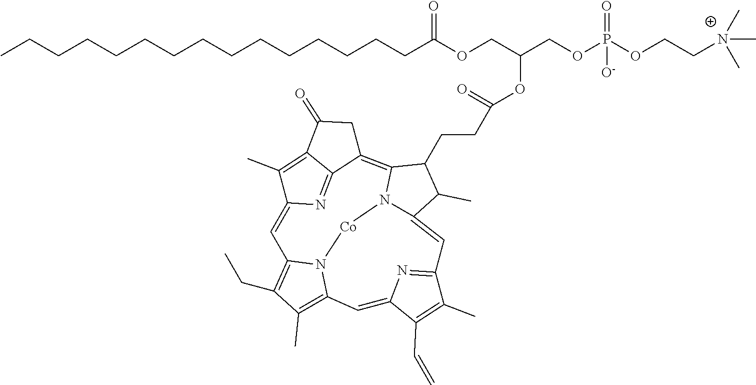

In an embodiment, the layer (monolayer or bilayer) has only CoPoP which has his-tagged presentation molecules embedded therein. In this embodiment, the only phospholipid in the layer is CoPoP (i.e., CoPoP is 100 mol %). In one embodiment, the layer has only CoPoP and porphyrin conjugated phospholipids (PoP), wherein layer has histidines embedded therein, with the histidines having a peptide or other presentation molecules (e.g., RBD sequences) attached thereto. In certain embodiments, there are no other phospholipids, but the layer may optionally contain sterols and/or PEG-lipid.

In an embodiment, in addition to the CoPoP, the bilayer also comprises phospholipids that are not conjugated to a porphyrin and, therefore, are not coordinated with Co. Such phospholipids may be referred to herein as “additional phospholipids”. The bilayer may also comprise one or more sterols and PEG or PEG-lipid. In one embodiment, the bilayer consists essentially of, or consists of CoPoP, phospholipids that are not conjugated to porphyrins, and optionally one or more sterols and/or PEG, wherein the PEG may be conjugated to lipid. In one embodiment, the only metal-PoP in the bilayer is CoPoP, where the layer has his-tagged presentation molecules embedded therein. In one embodiment, the only metal in the bilayer is Co.

In an embodiment, the bilayer of the liposomes comprises CoPoP and PoP. In addition to the CoPoP and the PoP, the bilayer can have additional phospholipids. The bilayer may further comprise one or more sterols and/or PEG. The PEG may be conjugated to a lipid. In one embodiment, the bilayer consists essentially of, or consists of CoPoP, PoP, additional phospholipids, and optionally one or more sterols and/or PEG, wherein the PEG may be conjugated to a lipid. In one embodiment, the only metal-PoP in the bilayer is CoPoP. In one embodiment, the only metal in the bilayer is Co.

In certain embodiments, the porphyrin is conjugated to the glycerol group on the phospholipid by a carbon chain linker of 1 to 20 carbons, including all integer number of carbons therebetween.

In various embodiments, in addition to the porphyrin conjugates disclosed herein, the bilayer of the liposomes also comprises other phospholipids. The fatty acid chains of these phospholipids may contain a suitable number of carbon atoms to form a bilayer. For example, the fatty acid chain may contain 12, 14, 16, 18 or 20 carbon atoms. In embodiments the bilayer comprises phosphatidylcholine, phosphatidylethanoloamine, phosphatidylserine and/or phosphatidylinositol.

The present bilayers may also comprise various sterols. The sterols may be

animal sterols or plant sterols. Examples of sterols include cholesterol, sitosterol, stigmasterol, and cholesterol. In embodiments, cholesterol may be from 0 mol % to 50 mol % or 0.1 to 50 mol %. In other embodiments, cholesterol may be present from 1 to 50 mol %, 5 to 45 mol %, or 10 to 30 mol %.

In certain embodiments, the bilayer further comprises poly-ethylene glycol (PEG)-lipid. The PEG-lipid can be DSPE-PEG such as DSPE-PEG-2000, DSPE-PEG-5000 or other sizes of DSPE-PEG. The PEG-lipid is present in an amount of 0 to 20 mol % including all percentage amounts therebetween to the tenth decimal point. The average molecular weight of the PEG moiety can be between 500 and 5000 Daltons and all integer values and ranges therebetween.

In an aspect, the disclosure provides a composition comprising liposomes or other structures of the present disclosure or a mixture of different liposomes or other structures. The compositions can also comprise a sterile, suitable carrier for administration to subjects including humans, such as, for example, a physiological buffer such as sucrose, dextrose, saline, pH buffering (such as from pH 5 to 9, from pH 7 to 8, from pH 7.2 to 7.6, (e.g., 7.4)) element such as citrate or phosphate. In an embodiment, the composition comprises at least 0.1% (w/v) CoPoP liposomes or his-tagged-CoPoP liposomes or other structures. In various embodiments, the composition comprises from 0.1 to 100 mol % CoPoP liposomes or his-tagged CoPoP liposomes or other structures such as bilayer coated nanoparticles. In an embodiment, the composition comprises from 0.1 to 99 mol % CoPoP liposomes having his-tagged presentation molecules associated therewith.

In an embodiment, the compositions of the present disclosure are free of maleimide or succinimidyl ester reactive groups. In one embodiment, the tagged molecule to be attached to the membrane does not have a non-natural amino acid.

The present disclosure also provides methods for using structures bearing the bilayers as described herein. In one embodiment, this disclosure provides a method of eliciting an immune response in a host. The immune response may generate antibodies. The method comprises administering to a subject a composition comprising a structure bearing CoPoP bilayers to which is conjugated a histidine tagged antigen. The compositions may be administered by any standard route of immunization including subcutaneous, intradermal, intramuscular, intratumoral, or any other route. The compositions may be administered in a single administration or may be administered in multiple administrations including booster shots. Antibody titres can be measured to monitor the immune response.

The data provided in the examples show that two separate RBD proteins, one produced in insect cells and one produced in mammalian cells both are potently adjuvanted by CoPoP liposomes, and with immunization produce orders of magnitude higher functional IgG response compared to other vaccine adjuvants that do not induce stable particle formation. It was surprising the RBD would work so effectively with CoPoP liposomes. For example, it is reported that other recombinant protein fragments such as malaria antigen fragment Pfs230 do not induce higher antibody levels compared to conventional vaccine adjuvants such as Alum (Huang et al., npj Vaccines volume 5, 23, 2020). Therefore the efficacy of an adjuvant seen here was surprising and could not be predicted.

The bilayers comprising CoPoPs can be prepared as follows. Freebase PoP can be produced by esterifying a monocarboxylic acid porphyrin such as pyropheophorbide-a with 2-palmitoyl-2-hydroxy-sn-glycero-3-phosphocholine (lyso-C16-PC), Avanti #855675P) using 1-Ethyl-3-(3-dimethylaminopropyl)carbodiimide and 4-dimethylaminopyridine in chloroform at a 1:1:2:2 lyso-C16-PC:Pyro:EDC:DMAP molar ratio by stirring overnight at room temperature. The PoP is then purified by silica gel chromatography. CoPoP can be generated by contacting porphyrin-phospholipid conjugate with a molar excess (e.g., 10-fold molar excess) of a cobalt salt (e.g., cobalt (II) acetate tetrahydrate) in a solvent (e.g., methanol) in the dark.

In an aspect, this disclosure provides vaccine compositions comprising the liposomes comprising CoPoP and his-tagged SARS-CoV-2 polypeptide sequences (such as the RBD portion of the Spike protein). The vaccine compositions may comprise one or more adjuvants. The vaccine compositions can comprise additives, such as diluents, adjuvants, excipients, or carriers. Such additives can be liquids, such as water, oils, saline, glucose or the like, and auxiliary, stabilizing, thickening, or lubricating agents, wetting or emulsifying agents, or pH buffering agents, gelling or viscosity enhancing additives, detergents and solubilizing agents (e.g., TWEEN® 20, TWEEN® 80 also referred to as polysorbate 20 or 80), anti-oxidants (e.g., ascorbic acid, sodium metabisulfite), preservatives (e.g., Thimerosal, benzyl alcohol), bulking substances (e.g., lactose, mannitol), flavoring agents, colors, and the like, depending upon the route of administration and the preparation desired. See Remington: The Science and Practice of Pharmacy (2012) 22nd Edition,. Non-aqueous solvents or vehicles can be used such as propylene glycol, polyethylene glycol, vegetable oils, such as olive oil and corn oil, gelatin, and injectable organic esters such as ethyl oleate. The formulations may be lyophilized and redissolved or resuspended just before use. The formulation may be sterilized by, for example, filtration through a bacteria retaining filter, by incorporating sterilizing agents into the compositions, by irradiating the compositions, or by heating the compositions.

The compositions can be introduced into a subject using any suitable administration route, including but not limited to parenteral, subcutaneous, intraperitoneal, intramuscular, intravenous, mucosal, topical, intradermal, and oral administration. Immunization can be done by way of a single dose or it can be done by multiple doses that are spaced apart. For example, an initial administration and subsequent booster doses can be used. The compositions can be administered alone, or can be co-administered or sequentially administered with other prophylactic (such as, for example, other immunogenic compositions) or therapeutic compositions (such as, for example, antiviral agents).

In one aspect, this disclosure provides a method of preventing a SARS-CoV-2 infection, or a severe (e.g., requiring hospitalization or virus-targeted medication) SARS-CoV-2 infection, also referred to herein as COVID-19 infection in a subject comprising administering to the subject an effective amount of a composition described herein. The vaccine composition may be administered once or multiple times. For example, the multiple doses of the vaccine composition may be administered with a suitable period in-between, such as days, weeks or months, and/or may be administered on an annual or any other periodic manner. This disclosure also provides a method of reducing the overall incidence of SARS-CoV-2 infection in a population comprising administering to a plurality of subjects in the population an effective amount of compositions described herein, whereby administration of the immunogenic compositions prevents the occurrence of SARS-CoV-2 infection in at least some of the subjects in the population such that overall incidence of SARS-CoV-2 infection in the population is reduced. Administration of this vaccine composition may prevent infections, prevent severe infections that need medication or hospitalization, and/or may prevent death attributable to the infection. Administration of this vaccine in a sufficient number of subjects in a given population may result in ‘herd’ immunity, thereby extending the protection to beyond those who receive this vaccine composition.

In an embodiment, this disclosure provides a method of eliciting an immune response in a subject comprising administering to a subject one or more doses of a liposomal composition described herein, wherein the liposomes comprise CoPoP and his-tagged SARS-CoV-2 polypeptide sequences (such as the RBD portion (e.g., SEQ ID NO:1 or variant thereof) of the Spike protein). The vaccine compositions may comprise one or more adjuvants, incorporated in the liposomes or separately present in the composition.

The dose of RBD needed for mice immunization are in a range from 100 ng to 5 μg, including all 0.1 ng values and ranges therebetween. The expected human dosing of the RBD may be 5-50 μg. In embodiments, the dose may be 0.1 μg to 1.0 mg. For example the dose may be 1 μg to 500 mg. In embodiments, the dose may be 1 to 100 μg or 10 to 50 μg or 5 to 10 μg or 1 to 10 μg or about 5 μg.

In embodiments, the present disclosure provides a liposome comprising:

a) a bilayer, wherein the bilayer comprises or consists essentially of:
i) phospholipid, and
ii) porphyrin having cobalt coordinated thereto forming cobalt-porphyrin; and

b) a polyhistidine-tagged SARS-CoV-2 molecule, wherein at least a portion of the polyhistidine tag resides in the hydrophobic portion of the bilayer and one or more histidines of the polyhistidine tag are coordinated to the cobalt in the cobalt-porphyrin, wherein at least a portion of the polyhistidine-tagged SARS-CoV-2 molecule is exposed to the outside of the liposome and the polyhistidine-tagged SARS-CoV-2 comprises an immunogenic sequence of a protein or polypeptide from SARS-CoV-2, and wherein the liposome encloses an aqueous compartment. In embodiments, the immunogenic sequence from SARS-CoV-2 polypeptides, such as the RBD sequence (such as SEQ ID NO:1) or a variant thereof, are the only amino acid sequences present in the bilayers. In some embodiments, other S protein sequences, or sequences from other proteins of the SARS-CoV-2 virus, and/or other immunogenic protein sequences may be present.

Administration of the present vaccine compositions to subjects may result in generation of an immune response in the subject. The immune response may be humoral response (e.g., the production of antibodies) or cellular response (e.g., the activation of T cells, macrophages, neutrophils, and/or natural killer cells) directed against the SARS-CoV-2 virus, or a protein target of the SARS-CoV-r virus (e.g., Spike protein). In embodiments, administration of the present vaccine compositions may elicit a protective immune response that can protect against SARS-CoV-2 infection or reduce the severity of the SARS-CoV-2 infection. A protective immune response may appear as one or more of the following—high titers of SARS-CoV-2 neutralizing antibodies, increased CD4+ or CD8+ cells, increased IFNγ, IL-2 and TNF

The present vaccine compositions may be administered to subjects who are at risk of contracting the SARS-CoV-2 virus (such as front-line health care workers), those with compromised immune system, or the population in general. It may be administered to a human of any age and gender. The vaccine compositions may be used in species other than humans in which the SARS-CoV-2 virus is capable of infecting.

The following Statements describe various examples and embodiments of the present disclosure:

Statement 1. A liposome comprising:

a) a bilayer, wherein the bilayer comprises:
i) phospholipid, and
ii) porphyrin having cobalt coordinated thereto forming cobalt-porphyrin; and

b) a polyhistidine-tagged presentation molecule, wherein at least a portion of the polyhistidine tag resides in the hydrophobic portion of the bilayer and one or more histidines of the polyhistidine tag are coordinated to the cobalt in the cobalt-porphyrin, wherein at least a portion of the polyhistidine-tagged presentation molecule is exposed to the3 outside of the liposome and the polyhistidine-tagged presentation molecule comprises an immunogenic sequence of a protein or polypeptide from SARS-CoV-2, and wherein the liposome encloses an aqueous compartment.

Statement 2. A liposome of Statement 1, wherein the protein is the RBD portion of the SARS-CoV-2 Spike protein.
Statement 3. A liposome of Statement 1, wherein the cobalt porphyrin is conjugated to a phospholipid to form a cobalt porphyrin-phospholipid conjugate.
Statement 4. A liposome of Statement 3, wherein the cobalt porphyrin-phospholipid conjugate makes up from 1 to 25 mol % of the bilayer.
Statement 5. A liposome of Statement 4, wherein the cobalt porphyrin-phospholipid conjugate makes up from 5 to 10 mol % of the bilayer.
Statement 6. A liposome of Statement 1, wherein the bilayer further comprises cholesterol and/or phosphatidylserine.
Statement 7. A liposome of Statement 1, wherein the polyhistidine-tag comprises 6 to 10 histidine residues.
Statement 8. A liposome of Statement 1, wherein size of the liposome is 50 nm to 200 nm.
Statement 9. A liposome of Statement 1, further comprising one or more adjuvants in the bilayer.
Statement 10. A liposome of Statement 9, wherein at least one of the adjuvants is attenuated lipid A derivative or phosphorylated hexaacyl disaccharide.
Statement 11. A liposome of Statement 10, wherein a second adjuvant is QS21.
Statement 12. A nanostructure comprising:

a) a core; and

b) a bilayer on said core, wherein the bilayer comprises:
i) phospholipid monomers, and
ii) porphyrin having cobalt coordinated thereto forming cobalt-porphyrin; and

c) a polyhistidine-tagged presentation molecule comprising an epitope from a microorganism, wherein at least a portion of the polyhistidine tag resides in the hydrophobic portion of the bilayer, one or more histidines of the polyhistidine tag are coordinated to the cobalt in the cobalt-porphyrin, and at least a portion of the polyhistidine-tagged presentation molecule is exposed on the outside of the nanostructure.

Statement 13. A nanostructure of Statement 12, wherein the core is a gold nanoparticle.
Statement 14. A nanostructure of Statement 12, wherein the microorganism is SARS-CoV-2.
Statement 15. A nanostructure of Statement 14, wherein the presentation molecule is a portion of the Spike protein from SARS-CoV-2.
Statement 16. A nanostructure of Statement 15, wherein the portion is the RBD region of Spike protein.
Statement 17. A method for generating an immune response in a host individual comprising administering to the individual a composition comprising the liposomes of any of the preceding Statements.
Statement 18. A method of Statement 17, wherein the individual is a human.
Statement 19. A vaccine composition comprising liposomes, wherein at least some of the liposomes comprise CoPoP, and a portion of the Spike protein of the SARS-CoV-2, wherein the portion of the Spike protein is covalently linked to poly-histidines, wherein the poly-histidine portion of the Spike protein is coordinated to the Co of the CoPoP portion such that at least a portion of the poly-his tag lies within the bilayer of the liposome and at least a portion of the Spike protein is exposed to the exterior of the liposome.
Statement 20. A vaccine composition of Statement 19, wherein the portion of the Spike protein is the RBD.

The following example is presented to illustrate the present disclosure. It is not intended to be limiting in any manner.

### EXAMPLE

This example illustrates embodiments of the present disclosure.

Results and Discussion

Intact, serum-stable RBD particles are obtained via admixing with CoPoP liposomes. Recombinant RBD proteins bearing a C-terminus His tag were obtained from mammalian (HEK293; Spike residues 319-541) and insect (sf9; Spike residues 330-530) expression systems. Liposomes containing CoPoP, as well as the adjuvants monophosphoryl lipid A (MPLA) and/or QS21 were mixed with the RBD for 3 hr at room temperature at a 4:1 mass ratio of CoPoP:protein and RBD binding to liposomes was then assessed. Control liposomes that lacked cobalt within the PoP molecule, but were otherwise identical, were also tested.

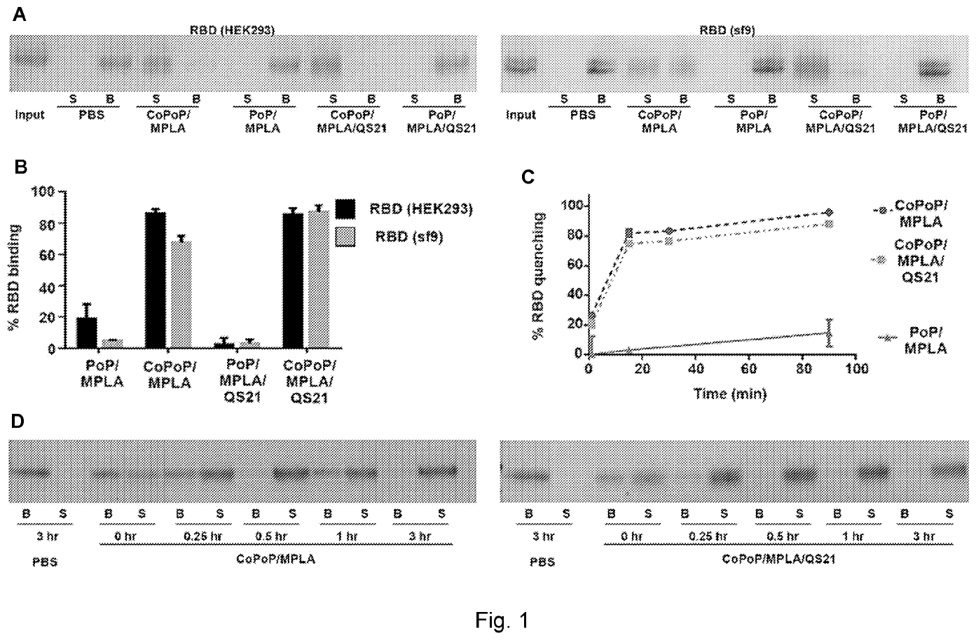

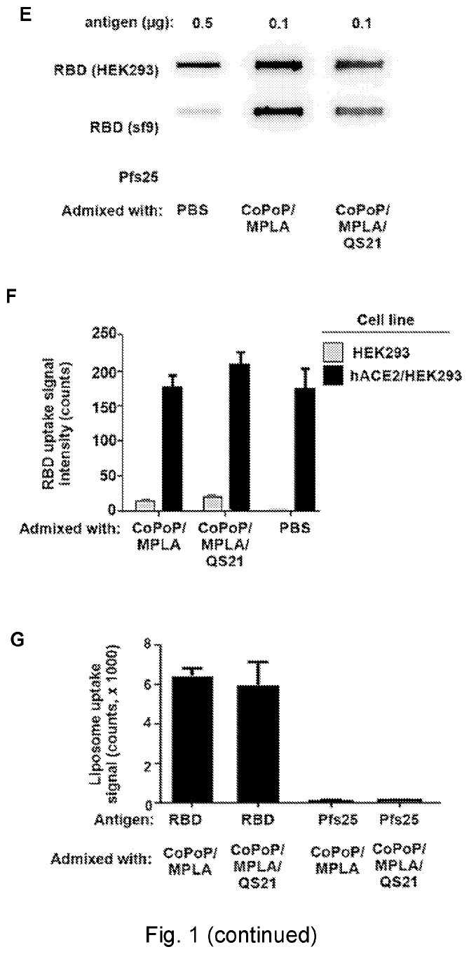

FIG. 1A, shows particle formation of the RBD based on a competition assay with Ni-NTA beads. The free protein is captured by the beads (“B”), whereas liposome-bound RBD does not and remains in the supernatant (“S”). The HEK293 and sf9 produced RBD exhibited nearly identical binding patterns, showing full binding to liposomes containing CoPoP, but virtually no binding to identical liposomes lacking cobalt, but still containing the PoP moiety. The presence of QS21 in the bilayer did not impact binding. The binding of the RBD to CoPoP, but not PoP, liposomes was also shown using an independent high speed centrifugation assay (FIG. 1B). A fluorescence resonance energy transfer assay was developed using a fluorescently-labeled RBD, which is quenched upon binding to liposomes due to energy transfer to the CoPoP chromophore. FIG. 1C shows the RBD particalization with most of the antigen forming particles within just 15 minutes of incubation. This result was confirmed using the Ni-NTA bead competition assay (FIG. 1D).

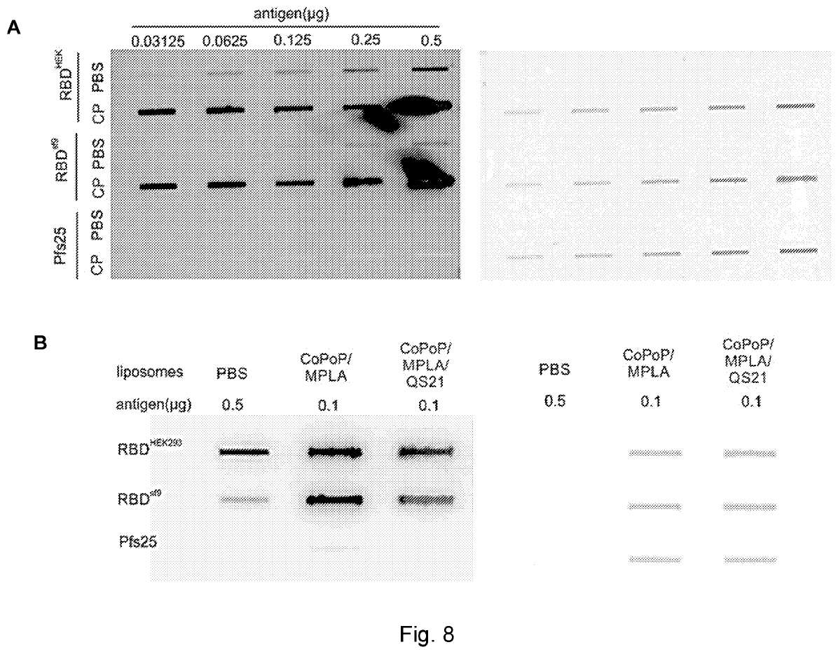

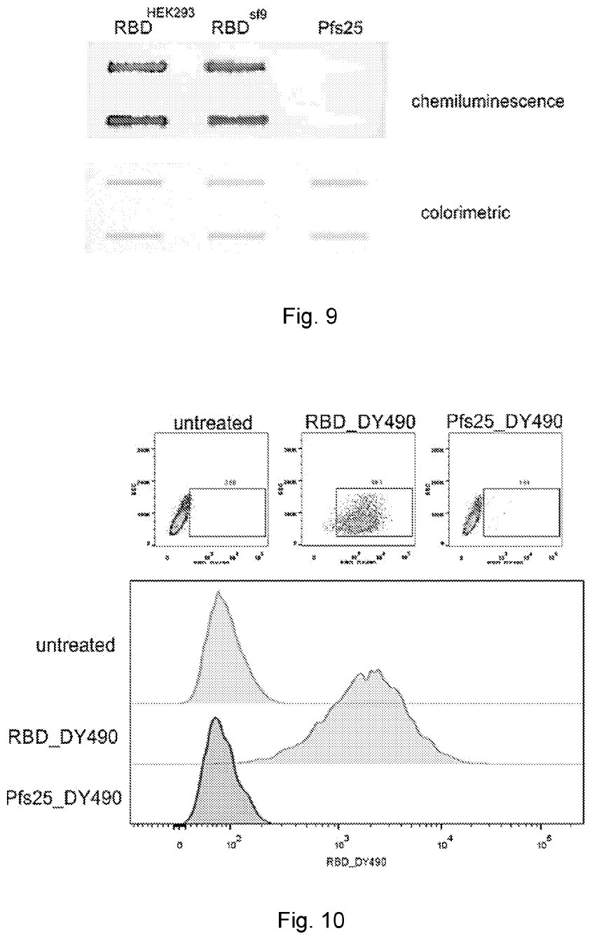

The conformational integrity of the RBD in particle form was next assessed. A slot blot was developed using ACE2, the binding target of the RBD, which was incubated with the RBD in either soluble or particle form, adsorbed on nitrocellulose. A secondary antibody then was used to detect ACE2. As expected, ACE2 did not recognize a Pfs25 control antigen included in the assay. Unexpectedly, ACE2 recognized the RBD more strongly in particulate form relative to soluble form, so that a reduced amount of particleized RBD needed to be used in the assay (FIG. 1E). The reason for this behavior is not clear, but the soluble RBD potentially adsorbs to the membrane in such a way that ACE2 becomes less accessible. Regardless, this result shows that the RBD maintains capacity for binding its target receptor in particle form. FIG. 8A compares the slot blot at varying doses of RBD in soluble or particle form. FIG. 8B shows ACE2 reactivity at a fixed RBD amount. Particleized RBD could also be recognized by the CR3022 neutralizing monoclonal antibody (FIG. 9), that is known to interact with the SARS-CoV-2 RBD.

When HEK293 cells that overexpress human ACE2 (HEK293/hACE2) were incubated with a fluorescently labeled RBD in both soluble and particle form, strong uptake was observed, as assessed by a whole cell lysate assay (FIG. 1F). The same cell line that lacked hACE2 expression exhibited minimal RBD uptake in all cases. A similar trend was observed using flow cytometry (FIG. 10). Not only was the RBD taken up preferentially by hACE2 expressing cells, but the CoPoP liposomes themselves showed strong uptake when decorated with the RBD (FIG. 1G). In contrast, liposomes decorated with Pfs25 showed minimal uptake. Overall, these biochemical data show that His-tagged RBD rapidly forms particles when incubated with CoPoP liposomes while maintaining structural integrity.

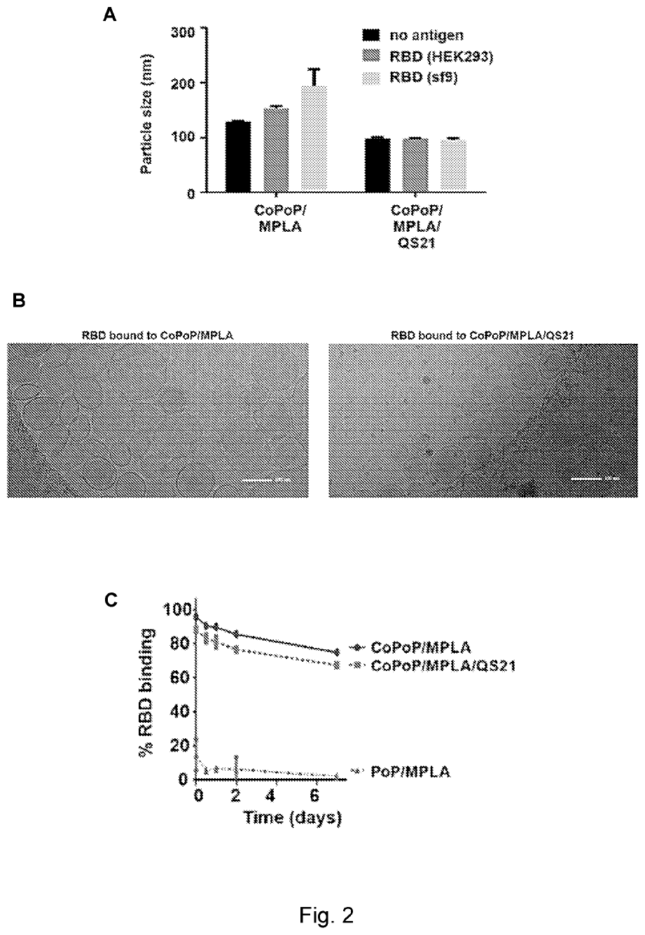

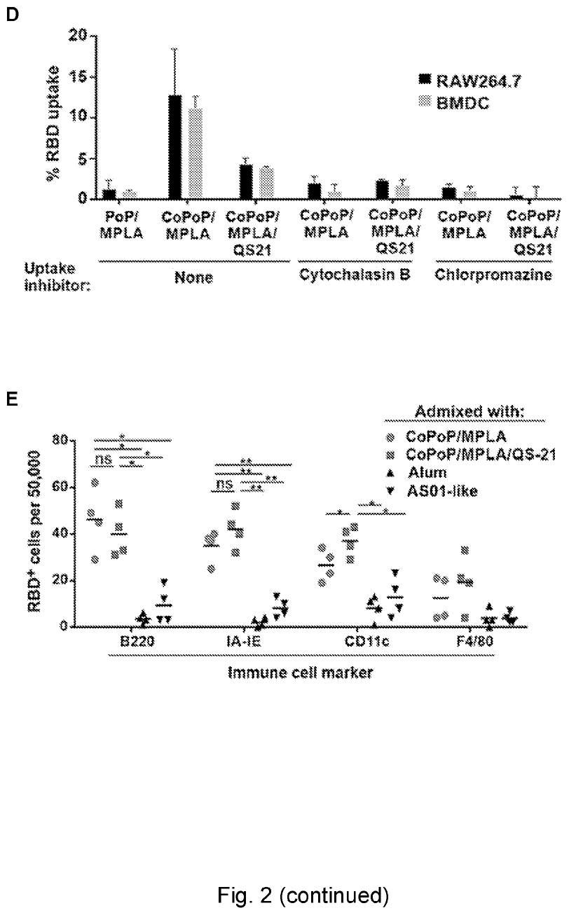

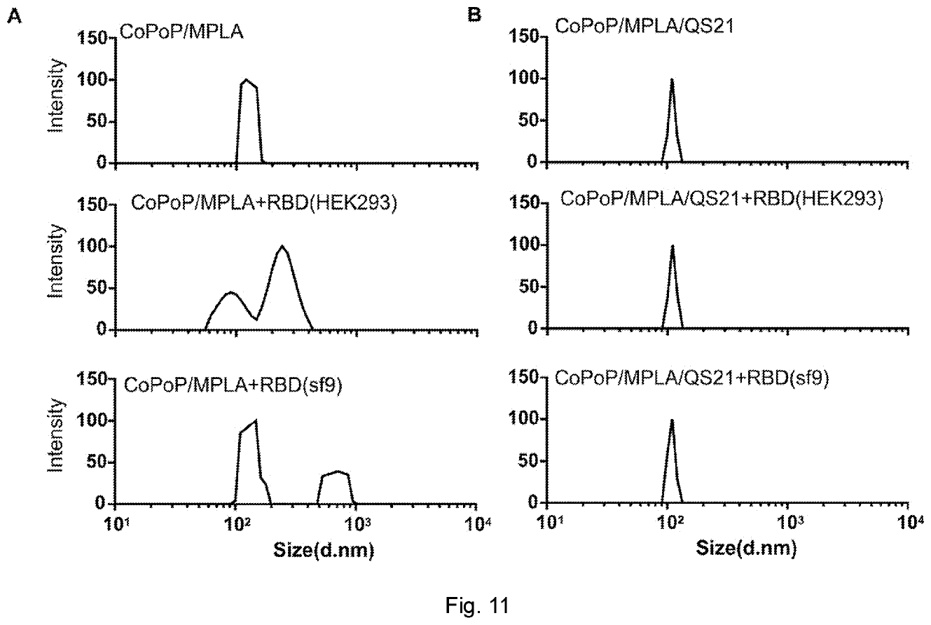

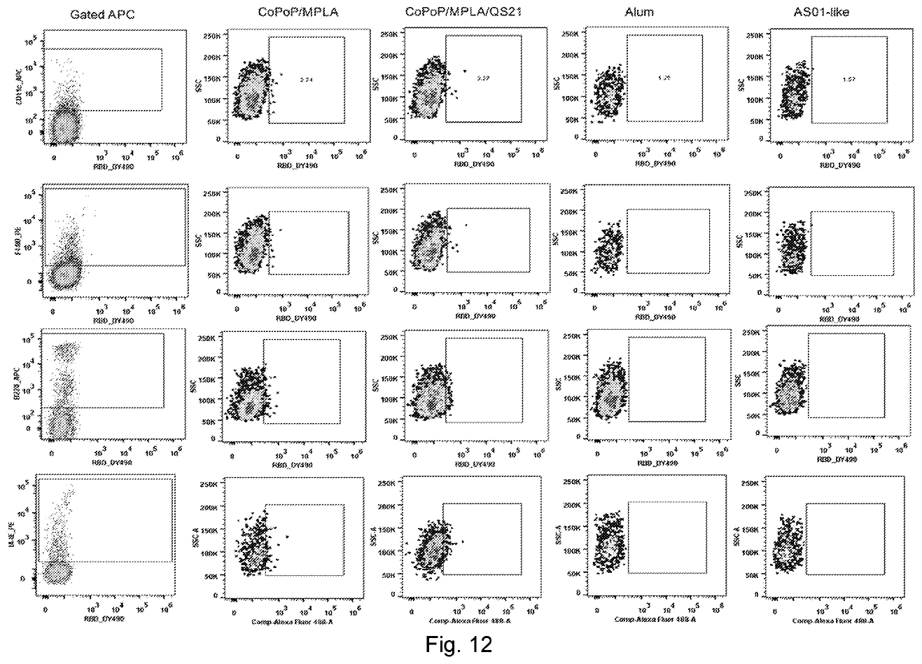

The nature of the particles themselves were next further assessed. There was a marginal increase in the size of CoPoP/MPLA liposomes following binding to the RBD, whereas CoPoP/MPLA/QS21 liposomes remained the same size with or without RBD (FIG. 2A, FIG. 11),). Neither liposome type exhibited aggregation upon antigen binding. Cryo electron microscopy revealed that the particleized RBD particles were spherical in nature with the QS21 liposomes showing slightly smaller size (FIG. 2B), consistent with the dynamic light scattering results. As shown in FIG. 2C, following particle formation, the RBD formed serum-stable antigen particles, based on a fluorescent quenching assay, indicating that the antigen was still maintained in the form of intact particles after 1 week incubation with 20% human serum at 37° C. To investigate the uptake of antigen particles by antigen-presenting cells (APCs), in vitro studies were performed with RAW264.7 murine macrophages and bone marrow derived dendritic cells (BMDC) obtained from outbred mice. APCs were incubated with fluorescently-labeled RBD and uptake was assessed (FIG. 2D). When the RBD was admixed with CoPoP liposomes, but not identical PoP liposomes (that did not induce particle formation), a higher RBD uptake by both macrophages and BMDCs was observed. However, when those cells were treated with cytochalasin B (a phagocytosis inhibitor) or chlorpromazine (an endocytosis inhibitor), particlized RBD uptake was inhibited. These data are consistent with a main mechanism for the efficacy of CoPoP liposomes to be related to improved uptake by APCs. To investigate whether enhanced uptake occurred in vivo, mice were immunized intramuscularly with the a fluorescently-labeled RBD admixed with various formulations, draining lymph nodes were collected 2 days later, and resident APCs were examined for RBD uptake by flow cytometry using markers B220 (for B-cells), F4/80 (for macrophages), CD11c (for dendritic cells), and I-A/I-E (for MHCII-expressing cells). As shown in FIG. 2E and FIG. 12, the RBD was significantly taken up by all the major types of APCs when presented in particulate form by admixing with CoPoP liposome. On the other hand, when adjuvanted with alum or non-particlizing AS01-like liposomes, minimal antigen uptake by the APCs was observed. The AS01-like liposomes were made in the lab and included synthetic MPLA and QS21 mimicking the formulation of the clinical liposomal adjuvant. These data show that following mixing with CoPoP, the RBD converted into 100 nm particles which were stable in serum and were preferentially taken up in vitro and in vivo by APCs.

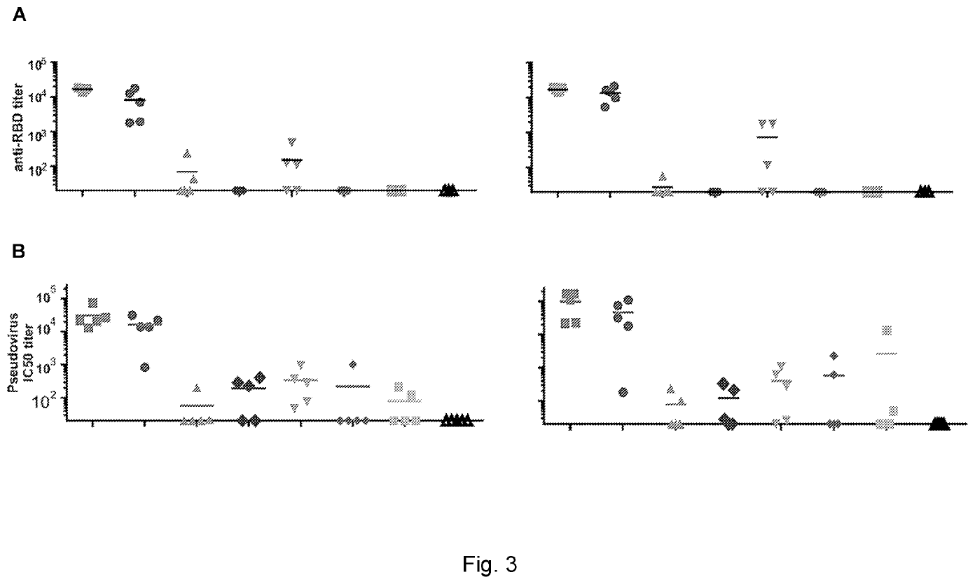

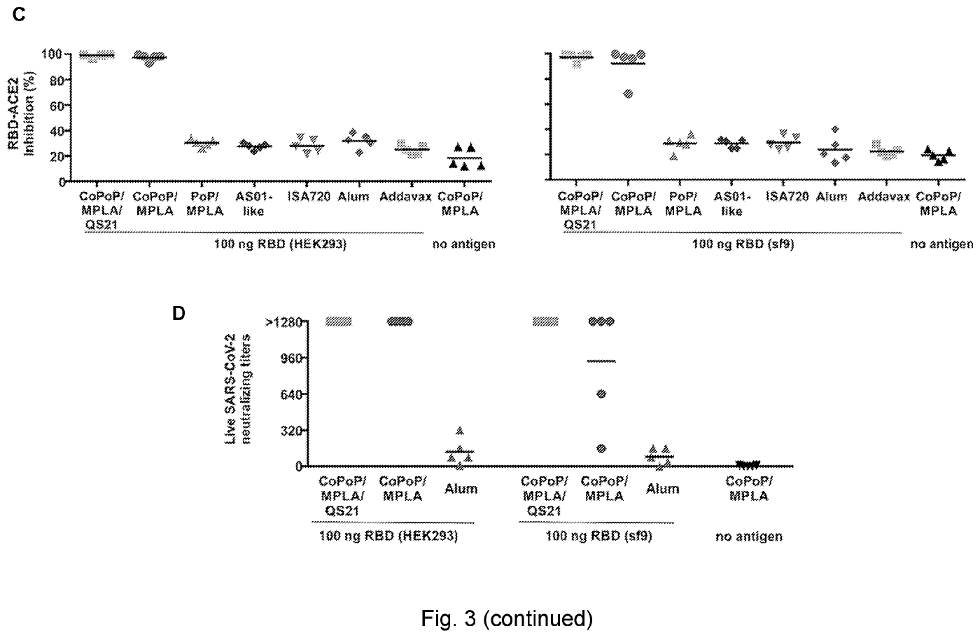

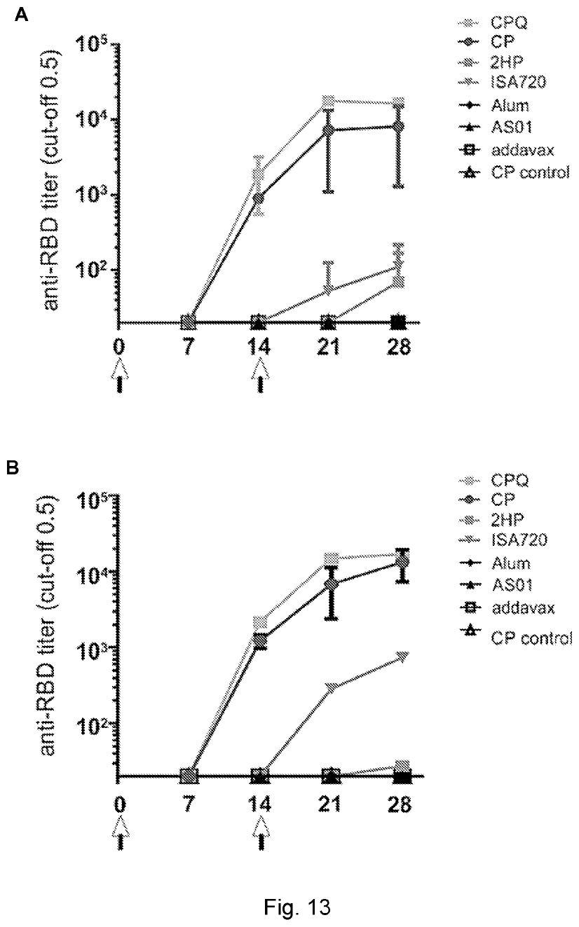

RBD potently induces neutralizing antibody responses in mice and rabbits in particle form. Mice were immunized intramuscularly with 100 ng of RBD (prepared from either insect or mammalian expression systems), admixed prior to immunization with commercially obtained vaccine adjuvants Alum, Montanide ISA720, or Addavax, or alternatively, the lab-made CoPoP/MPLA, CoPoP/MPLA/QS21, PoP/MPLA, or AS01-like liposomal adjuvants. No additional purification was carried out after mixing antigen and adjuvants. A significant increase in RBD-specific IgG was observed with the CoPoP adjuvants prior to boosting on day 14 (FIG. 13). FIG. 3A shows the day 28 end point anti-RBD titer, demonstrating that admixing with CoPoP increased the anti-RBD titer levels compared to other adjuvants, as well as PoP/MPLA liposomes (which lack cobalt but are otherwise identical to CoPoP/MPLA liposomes) by 2-3 orders of magnitude. Responses induced by mammalian- and insect-produced RBD were similar. The adjuvant itself without the inclusion of the antigen did not induce any RBD antibodies. The magnitude of the antibody response for the CoPoP samples provides evidence of the advantage of delivering RBD in a particle format, likely due to enhanced uptake by APCs (FIG. 2E).

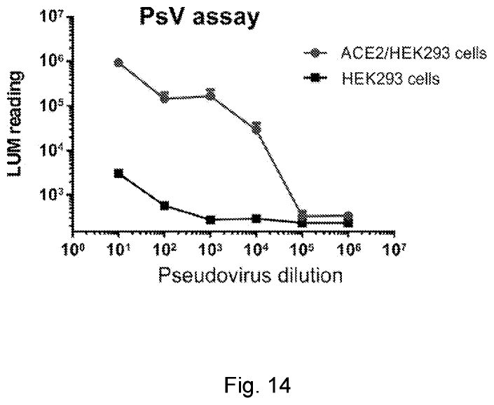

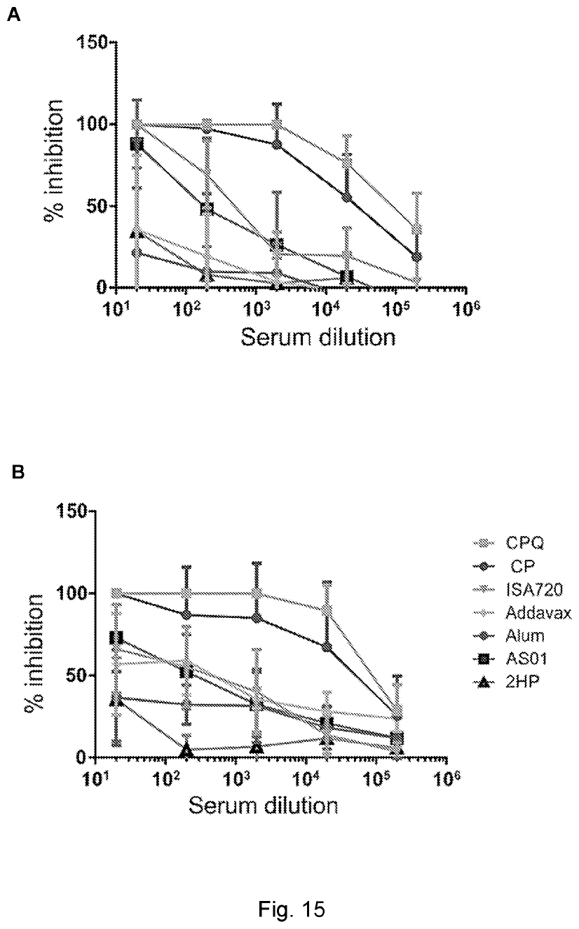

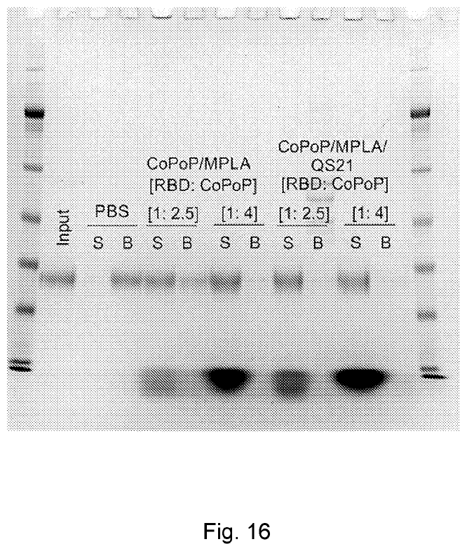

Antibody function was initially assessed with a pseudovirus (PsV) assay. A murine leukemia virus-based PsV expressing luciferase and gag/pol proteins pseudotyped with the S protein of SARS-CoV-2 (SARS2) was produced in HEK293T cells and was found to selectively enter cells expressing hACE2 (FIG. 14). As shown in FIG. 3B, compared with other vaccine adjuvant groups, the sera from the RBD/CoPoP group potently inhibited viral entry. The NT₅₀ (50% of neutralizing antibody titers) of the mice immunized with the mammalian produced RBD admixed with CoPoP/MPLA liposome was 16,430, whereas the NT₅₀ with CoPoP/MPLA/QS21 was 30,827. These NT₅₀ values were orders of magnitude greater than most other the adjuvants including ISA720 (NT₅₀: 339); Addavax (NT₅₀: 78); PoP/MPLA (NT₅₀: 56.6); Alum (NT₅₀: 219); ASO1 (NT₅₀: 191). These PsV neutralizing titers were similar for the insect-produced RBD. FIG. 15A and FIG. 15B show the full PsV entry inhibition curves for mammalian and insect produced RBD.

A SARS-CoV-2 Surrogate Virus Neutralization Test (sVNT) was used to

further assess the nature of the functional antibodies. The sVNT assay is an in vitro, cell-free method that detects antibodies that block the interaction of hACE2 and the RBD and has been used to predict neutralizing antibody titers in clinical specimens. As shown in FIG. 3C, at 100-fold dilution, post-immune sera for CoPoP-immunized mice inhibited 99% of the interaction between RBD and ACE2. In contrast, all the other vaccine adjuvants produced an inhibition that was at the baseline level of approximately 30%, the same level of serum of mice that did not receive any RBD-immunogen at all. Again, sVNT results were similar between mammalian and insect produced RBD.

Next, a live virus neutralization test (VNT) was carried out with the SARS-CoV-2 strain, USA-WA1/2020. Sera from mice immunized with the mammalian or insect produced RBD admixed with CoPoP liposomes prevented pathogenic cellular infection at 1:1,280 diluted sera, the highest dilution assessed (FIG. 3D). Convalescent sera therapy recommends the use of sera with a 1:160 VNT titer, thus nanogram dosing in mice induced strongly functional antibodies. When admixed with Alum, the RBD induced antibodies with limited capacity for virus neutralization. Taken together, a range of antibody tests show that the RBD benefits greatly admixing with a vaccine adjuvant that induces particle formation.

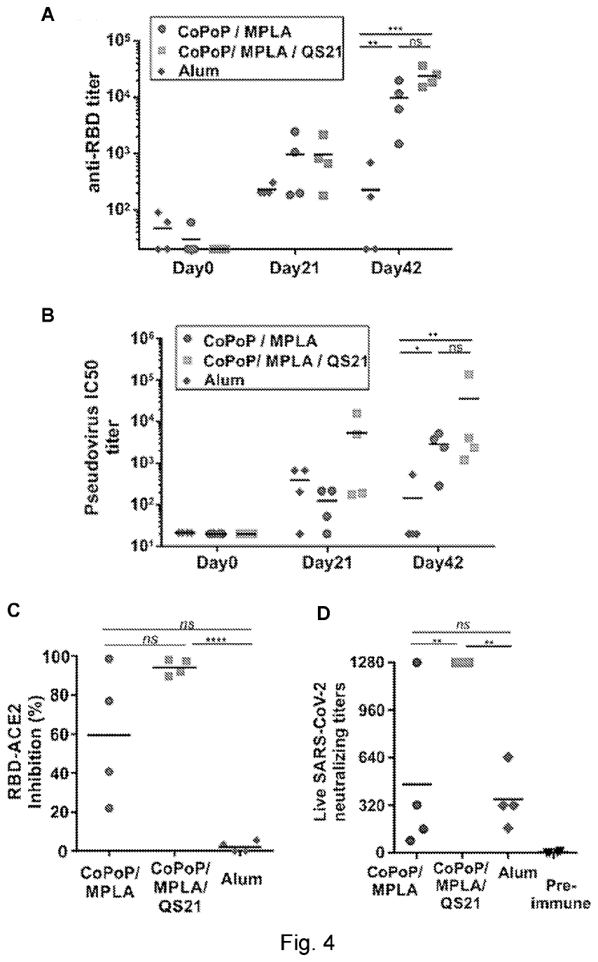

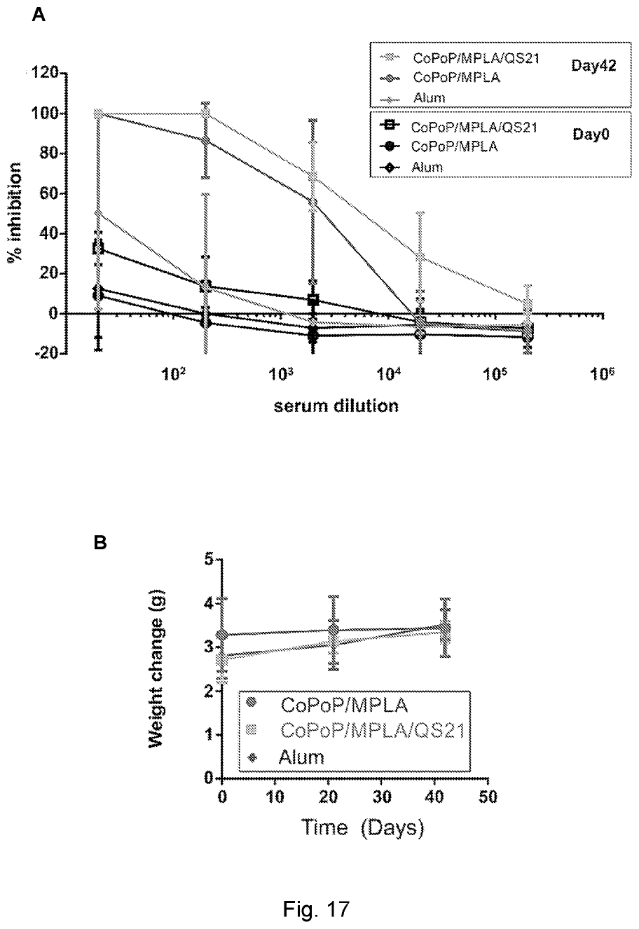

Next, to assess RBD immunization in another animal species, rabbits were immunized with a human relevant 20 μg dose of RBD admixed with either CoPoP liposomes or Alum, intramuscularly, on day 0 and day 21. The post-immune sera showed anti-RBD IgG presence on day 21 followed by a boosting effect that led to the final day 42 antibody levels to be approximately 10-fold higher for CoPoP compared to Alum (FIG. 4A). This is less than the 2-3 fold order of magnitude enhancement over Alum observed in mice. As shown in FIG. 4B and FIG. 17, high pseudovirus neutralization activity occurred in rabbits immunized with CoPoP liposomes. Interestingly, inclusion of QS21 in the liposomes significantly enhanced neutralization titer after boosting. Again, this result differs from the mouse data, where QS21 benefits appeared more modest. Similar results were observed in the sVNT assay where QS21 inclusion enhanced the blocking of the interaction between ACE2 and the RBD (FIG. 4C). When live SARS-CoV-2 virus neutralization was assessed with VNT, post-immune sera from rabbit immunized with CoPoP/MPLA/QS21 liposomes prevented infection at the highest dilution tested (1:1,200), whereas only 1 of the 4 rabbit immunized with CoPoP/MPLA liposomes had neutralizing antibodies with comparable efficacy (FIG. 4D). The reason for the enhancement of QS21 in rabbits requires further investigation, but could relate to the higher antigen dose used in rabbits, or more likely, immunological differences between species. Examination of the mouse antibody data reveals that in general the QS21 CoPoP post-immune sera also generally had improved viral inhibition function, although the enhancement was subtle.

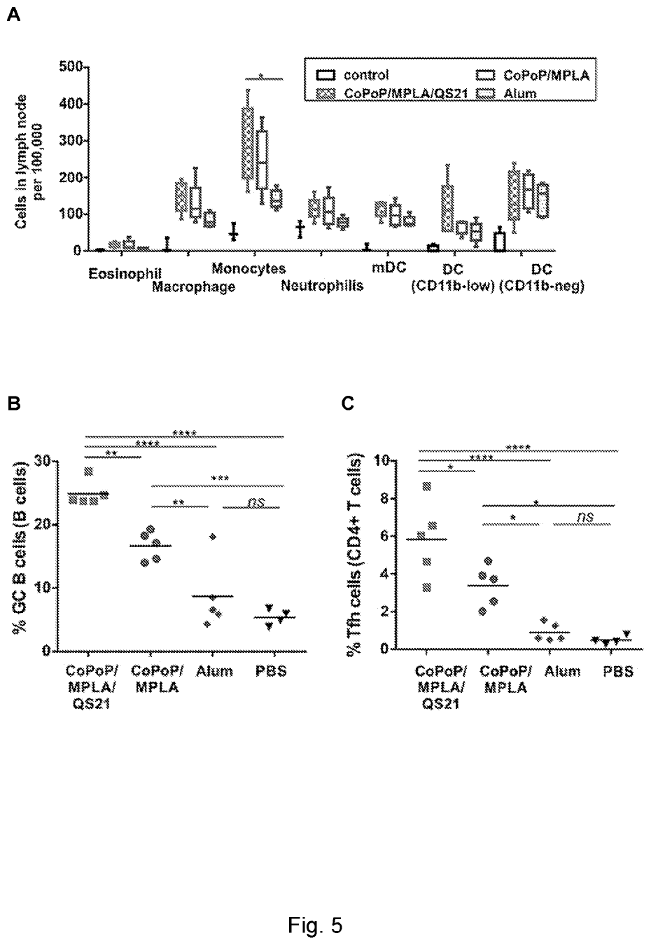

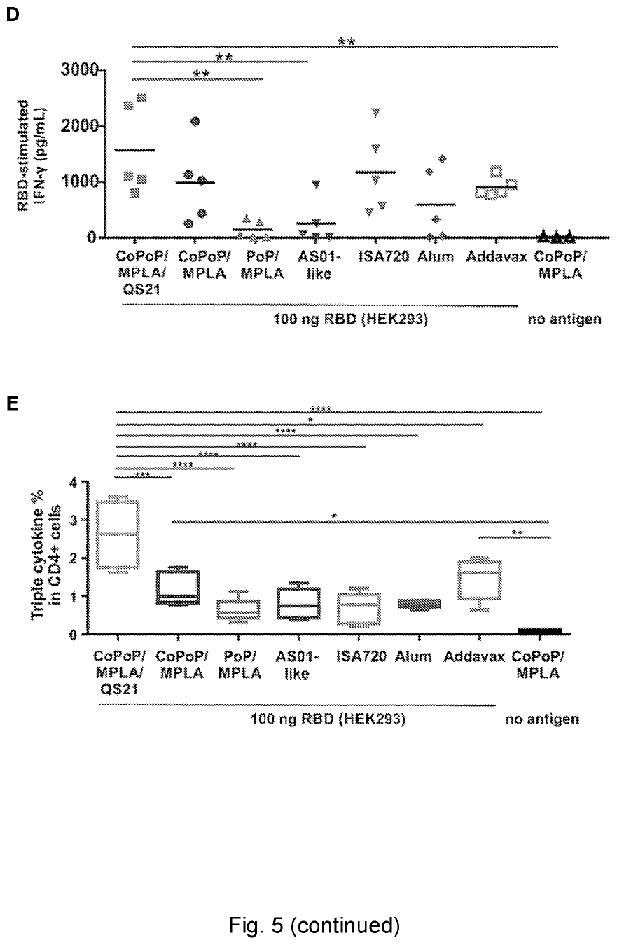

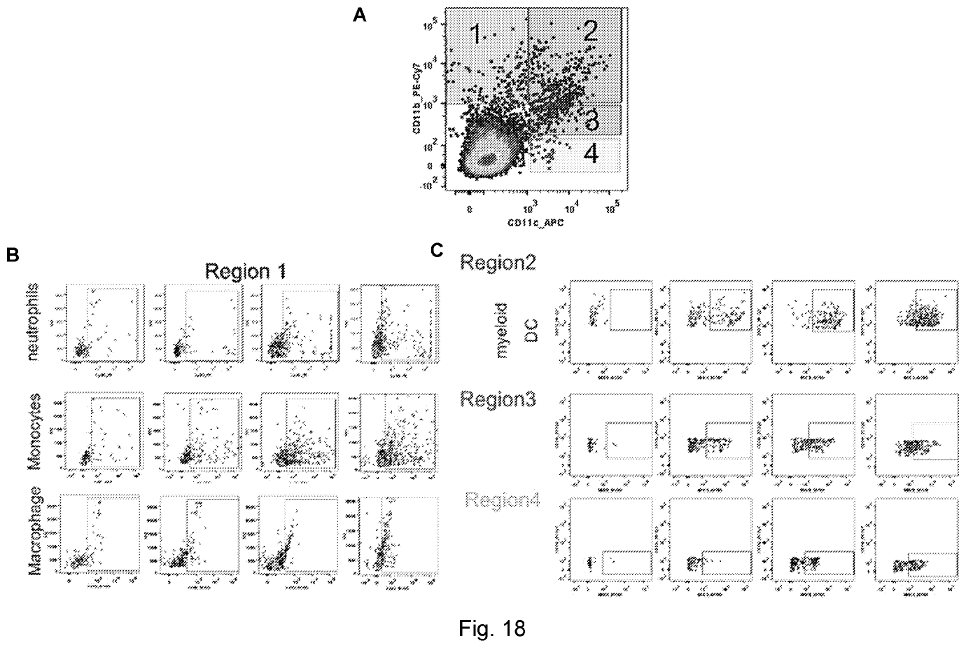

Cellular and humoral response. Immune cell recruitment was assessed 2 days after mouse intramuscular immunization with 100 ng of RBD admixed with CoPoP/MPLA liposomes, CoPoP/MPLA/QS21 liposomes, Alum or PBS. Flow cytometry was used to discriminate various cells in the draining lymph nodes. As shown in FIG. 5A and FIG. 18, CoPoP/MPLA liposomes and CoPoP/MPLA/QS21 liposomes induced enhanced recruitment of macrophages and monocytes compared to Alum. An increased level of CD11b⁻ DCs was shown for all the adjuvant groups. Cd11b⁻ DC cells play a role in cellular adaptive immune responses. An increased level of CD11b^{low} DCs was observed in mice treated with CoPoP/MPLA/QS21, but not CoPoP/MPLA liposomes nor Alum. Therefore, a second factor by which CoPoP appears to lead to potent immunization, besides improving antigen delivery to APCs, is by enhanced recruitment of APCs.

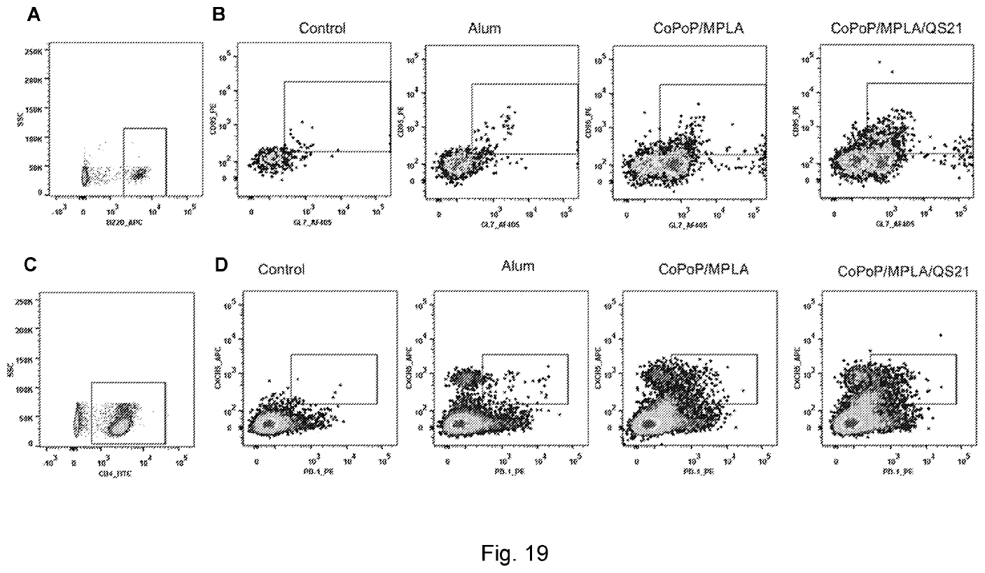

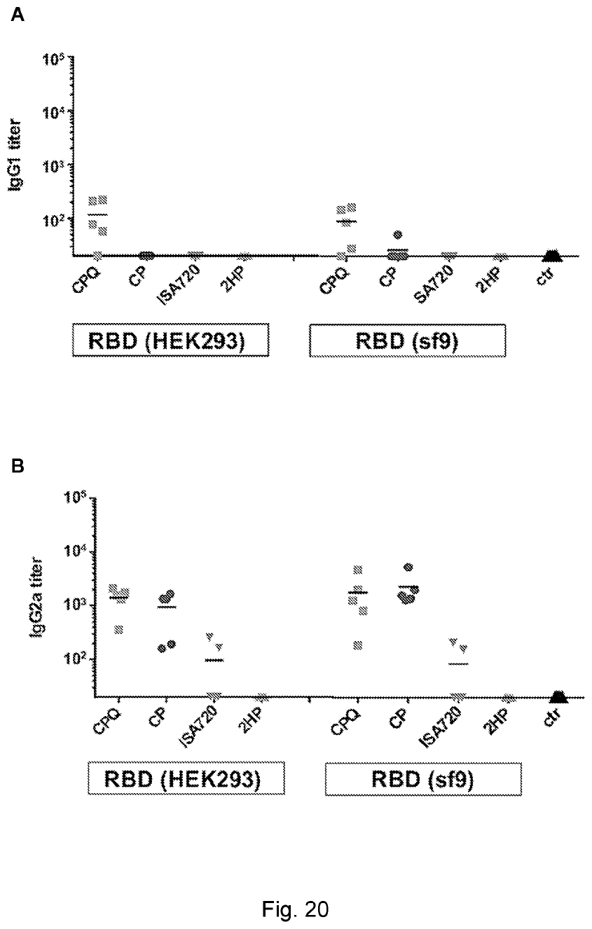

Germinal center (GC) formation was assessed following immunization. CoPoP liposomes enhanced the population of GC B cells, as well as the population of T follicular helper cells (Tfh cells). QS21 induced a higher degree of GC formulation (FIGS. 5B and 5C; FIG. 19). Tfh cells play a major role in protective immunity by helping B cells generate neutralizing antibody. This result may account for the enhanced immunity of the QS21-containing liposomes observed in rabbits. FIGS. 20A and 20B show that mice immunized with CoPoP/MPLA, as well as CoPoP/MPLA/QS21 liposomes elicited higher levels of IgG2a antibodies than IgG1 antibodies, suggesting the immune response was biased towards a Th1 response.

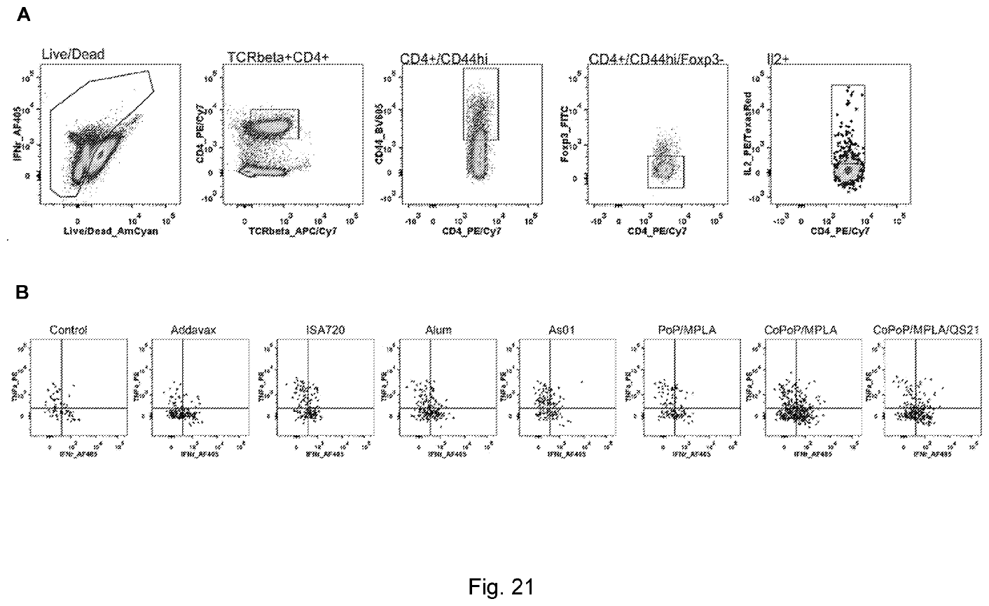

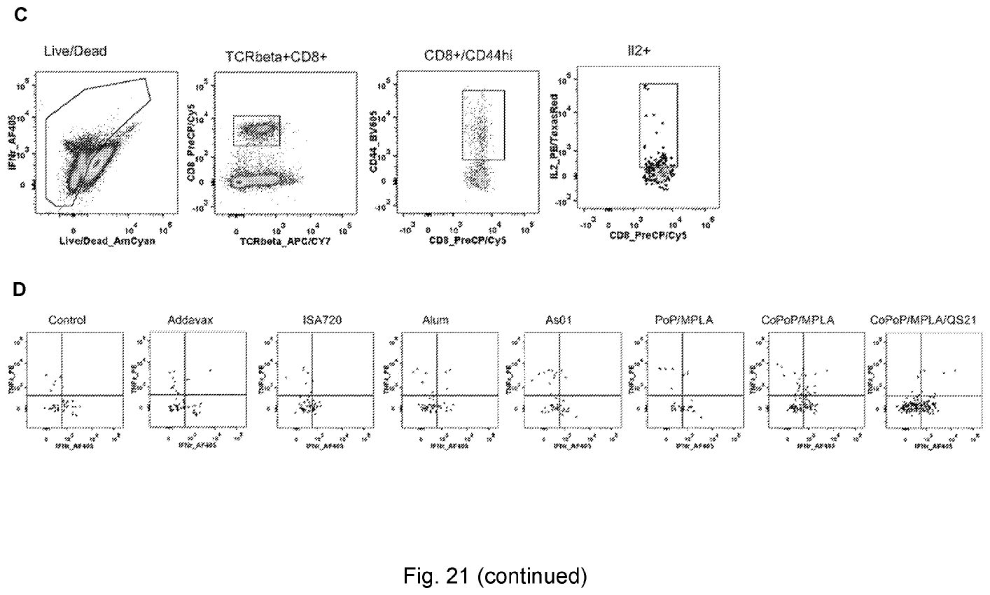

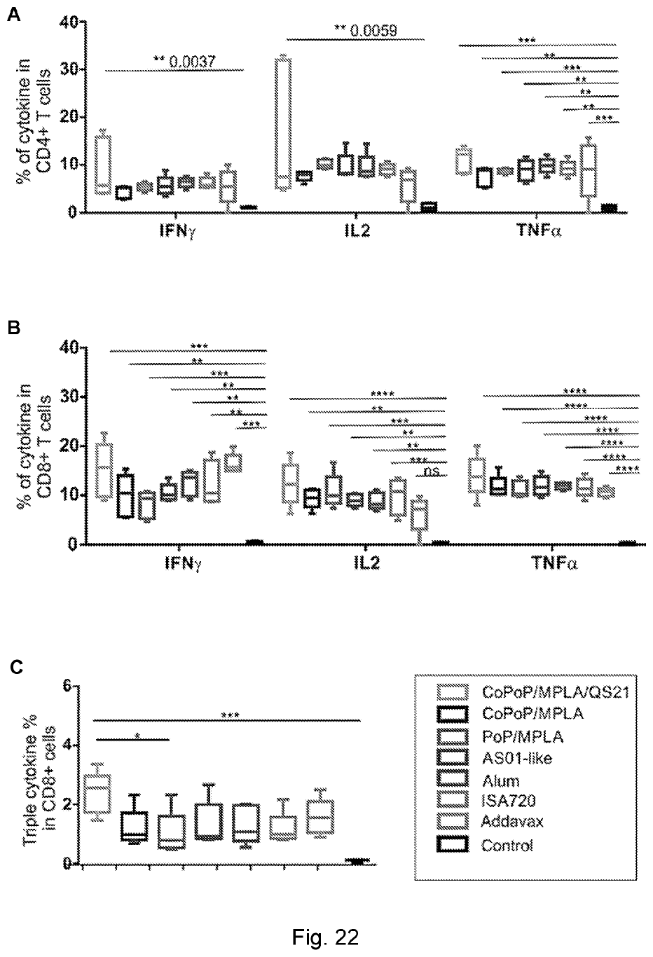

Following day 0 and day 14 RBD immunization, splenocytes were isolated and assessed for induction of interferon gamma (IFNγ) secretion following exposure to the antigen. Splenocytes from the mice immunized with CoPoP secreted higher levels of IFNγ relative to other adjuvants. This reflects higher antigen-specific T cell populations that were produced with the CoPoP adjuvant. Again, QS21 addition appeared to be beneficial in enhancing T cell responses. Polyfunctional T cells which express multiple cytokines have been shown as a protective immunity in viral infection. Antigen-specific CD4⁺ T cells which secrete IFNγ, IL-2 and TNFα are desirable to protect against infection. Antigen-specific CD8⁺ and CD4⁺ T cells that secrete IFNγ and TNFα indicate a memory phenotype and might lead to long-term protection for SARS-CoV. In order to address induction of polyfunctional T cells, splenocytes were collected from immunized mice, followed by RBD stimulation in vitro. The cells were assessed with flow cytometry, first gating live/dead cells, followed by gating TCRβ⁺CD4⁺CD44^{hi}Foxp3⁻ for memory CD4⁺ T cells and TCRβ⁺CD8⁺CD44^{hi} for memory CD8⁺ T cells (FIG. 21). As shown in FIG. 22A-B, single cytokine-producing populations in CD4⁺ T cells and CD8⁺ T cells were observed for IFNγ, IL-2 and TNFα. Later, cells were gated to assess all three cytokines, showing that splenocytes from mice immunized with the RBD and CoPoP/MPLA/QS21 generated stronger triple cytokine-producing populations in CD4⁺ T cells (FIG. 5D), as well as CD8⁺ T cells (FIG. 22C).

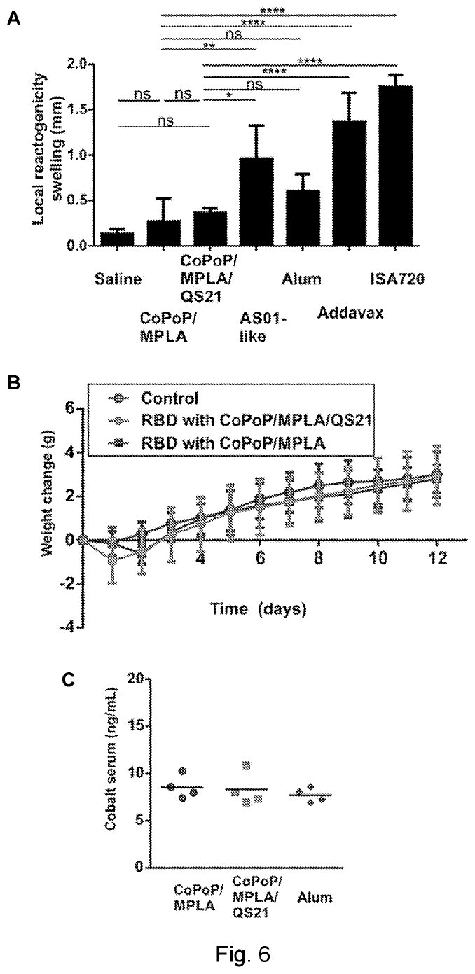

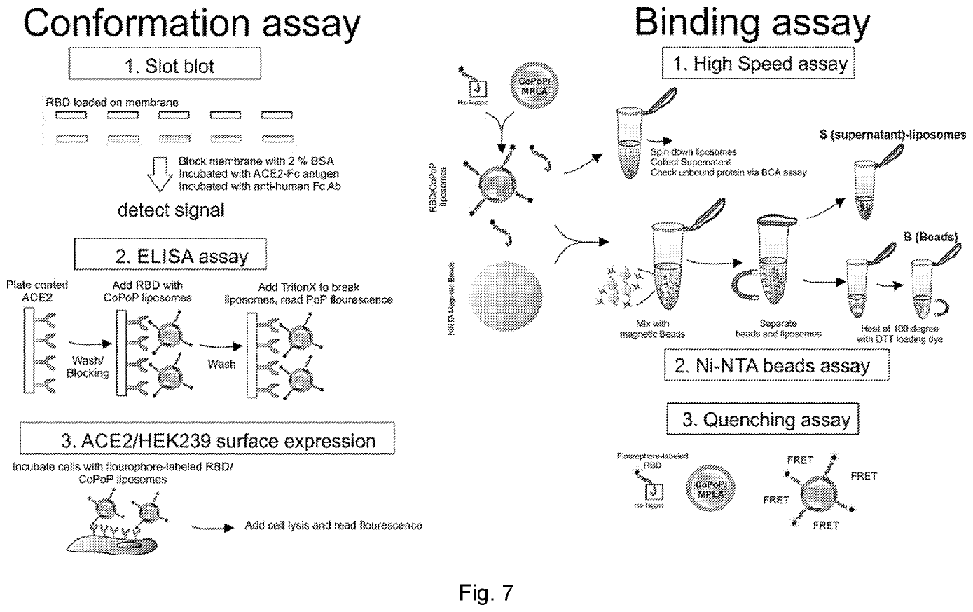

Safety of RBD immunization with CoPoP. Local reactogenicity of RBD admixed with various adjuvants was assessed in mice using a footpad swelling assay following a single intradermal vaccine injection. CoPoP/MPLA liposomes produced the least amount of local reactogenicity of all the adjuvants assessed, which included AS01-like liposomes, Alum, Addavax and ISA720 (FIG. 6A). The MPLA and QS21 content of CoPoP liposomes used throughout all the experiments in this work is 60% less than the AS01-like formulation, which may contribute to the relatively decreased reactogenicity.

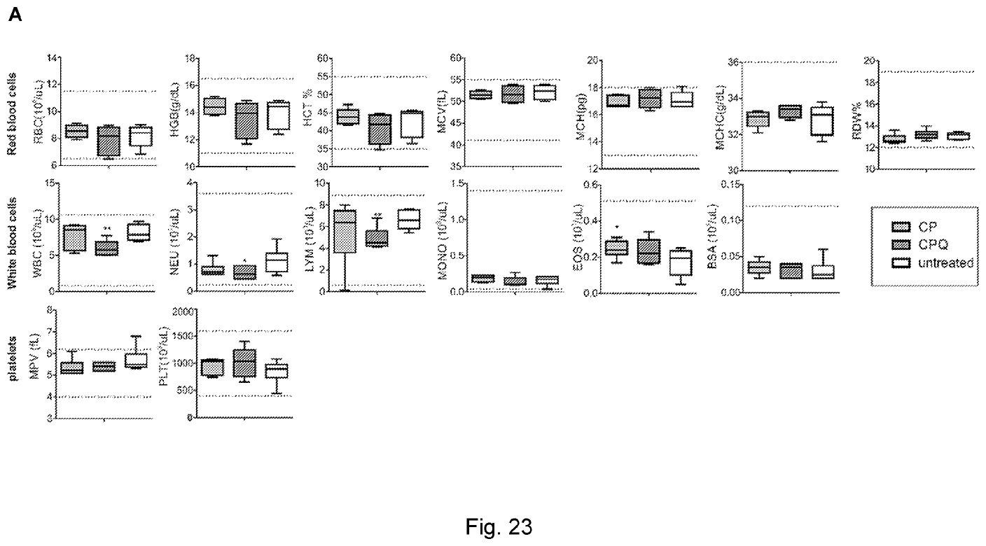

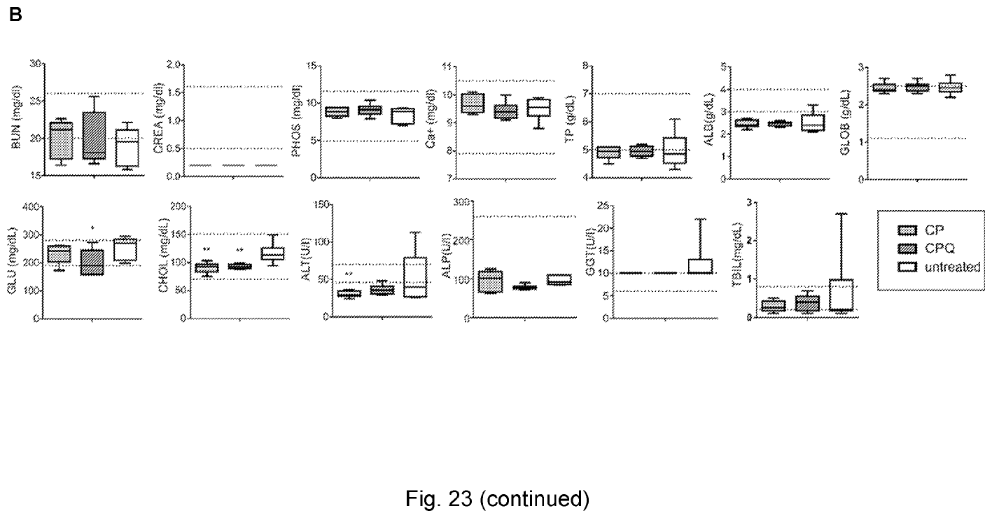

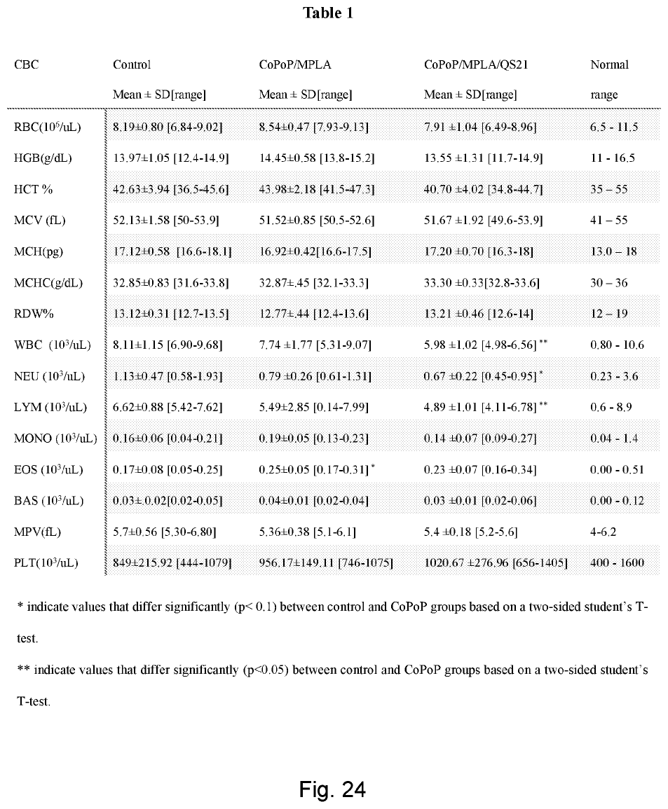

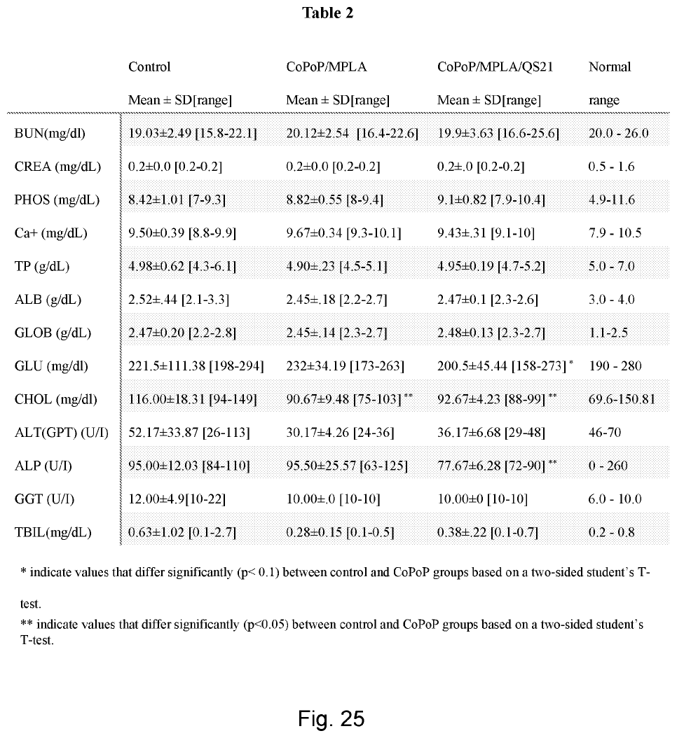

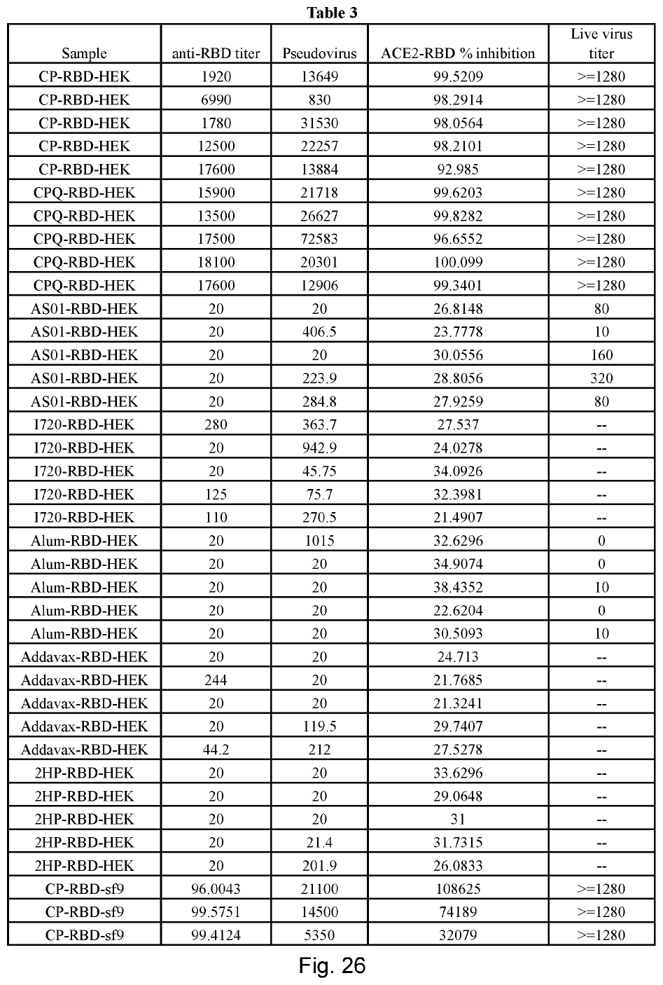

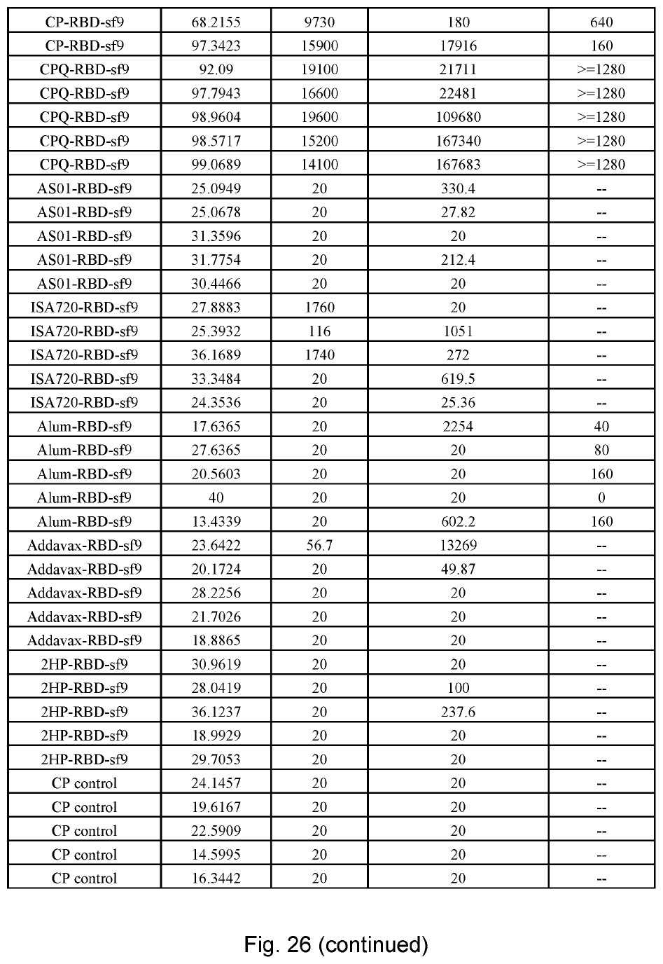

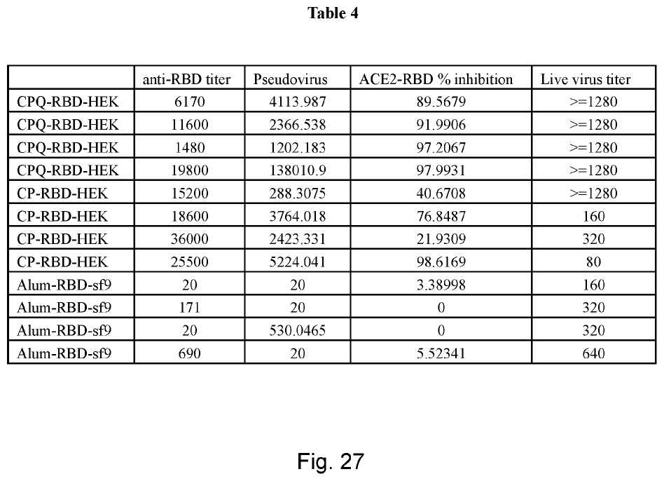

Safety studies were carried out in mice using 1 μg RBD with CoPoP/MPLA or CoPoP/MPLA/QS21 liposomes (along with an MPLA and QS21 dose of 1.6 μg). This is a dose tenfold higher than that used for the immunogenicity studies. Mice immunized with CoPoP exhibited normal weight gain compared to untreated mice (FIG. 6B). A complete blood cell count (FIG. 24 (Table 1), FIG. 23A) and serum chemistry panel analysis (FIG. 25 (Table 2), FIG. 23B) two weeks following treatment revealed normal ranges for all of the parameters assessed. While most parameters assessed were not different between control and immunized mice there were differences in white blood cells (WBC), neutrophils (NEU), lymphocytes (LYM), glucose (GLU), cholesterol (CHOL) and alkaline phosphatase (ALP) levels when comparing CoPoP/MPLA/QS21 to untreated mice, and differences of the EOS and CHOL levels for CoPoP/MPLA-treated compared to untreated mice. However, all measurements were within the normal range for the tests and thus we do not consider the measurements to reflect any sign of toxicity, even at the elevated dosing use. The use of cobalt in CoPoP is a potential concern for a vaccine, although it is worth noting that vitamin B12, a cobalt tetrapyrrole has been shown to be safe in humans with 5 gram intravenous doses, a level approximately 10,000× higher than anticipated for CoPoP human dosing. Following mouse immunization, serum cobalt levels were not elevated relative to mice that received the RBD with Alum (thus lacking any exogenous cobalt) (FIG. 6C).

Materials and Methods

Materials: His-tagged RBD expressed in the human embryonic kidney 293 cells (HEK293) cell line was purchased from RayBiotech (Cat #230-01102-100) and His-tagged RBD expressed in sf9 cells was purchased from Genscript (Cat #Z03479). CoPoP and PoP were produced, as previously described.

PoP synthesis: PoP is the precursor to CoPoP and is synthesized with a modified procedure from the initial description from Lovell et al. (Nat Mater. 2011; 10(4): 324-32). Both the C16 phosphatidylcholine lysolipid (Cat ##855675P; Avanti Polar Lipids, Alabaster, AL) and the porphyrin (Cat #P109-0014; Proactive Molecular Research, Alachua, FL) are commercially available with certificates of analysis, respectively. The porphyrin lipid esterification reaction utilizes carbodiimide chemistry; is purified via silica gel column chromatography; lyophilized into a powder; and is aliquoted and stored at −20° C. Purity is assessed using HPLC and identity is confirmed with mass spectrometry and NMR. Our current standard operating procedure, with batch record form, is capable of producing PoP at a 4 gram scale, with a yield of >85% and purity of >95%.

CoPoP synthesis: CoPoP is generated with a modified procedure from what we have reported (Shao et al., Nat Chem. 2015; 7(5): 438-446). In brief, a 30 molar excess of cobalt nitrate (Cat #36418; Alfa Aesar) is chelated by PoP (2 g) by stirring overnight in methanol (100 mL) at room temperature. Unchelated cobalt is removed by liquid-liquid extraction using a 1% methanol/water and chloroform solution to extract the lipid and water to remove the excess cobalt. Chloroform is removed from the liquid-liquid extraction purified CoPoP using rotary evaporation. The solid CoPoP is redissolved in tert-butanol and lyophilized for storage at −20° C. as a dark green powder. The current 3 gram yield of CoPoP with existing protocol is sufficient for 75,000 human doses, for a 10 μg dose with a 4:1 ratio of CoPoP:antigen. The solid CoPoP is redissolved in tert-butanol and lyophilized for storage at −20° C. as a dark green powder. CoPoP purity is assessed by HPLC and identity confirmed with mass spectrometry (Shao et al., Nat Chem. 2015; 7(5): 438-446).

The following adjuvants were obtained: Montanide ISA720 (SEPPIC) and Alhydrogel 2% aluminum gel (Accurate Chemical and Scientific Corporation; Cat #A1090BS), Addavax (InVivoGen Cat #vac-adx-10). The following lipids were used: 1,2-dipalmitoyl-sn-glycero-3-phosphocholine (DPPC, Corden Cat #LP-R4-057), 1,2-Dioleoyl-sn-glycero-3-phosphocholine (DOPC, Avanti cat # 850375), cholesterol (PhytoChol, Wilshire Technologies), synthetic monophosphoryl Hexa-acyl Lipid A, 3-Deacyl (PHAD-3D6A, Avanti Cat #699855). QS21 was obtained from Desert King. Granulocyte-macrophage colony-stimulating factor (GM-CSF) was obtained from Shenandoah Biotechnology (GM-CSF; cat #200-15-AF). Cytochalasin B was obtained from ThermoFisher Scientific (cat #14930-96-2). Antibodies for flow cytometry: For immune cell recruitment in lymph nodes, the following antibodies were obtained from Biolegend: CD11c-APC Cy7 (Clone: N418; Cat #117323; Lot B237078), CD3 PerCP/Cy5.5 (Clone: 17A2; Cat #100217; Lot B233419), I-A/I-E Alex Fluor 700 (Clone: M5/114.15.2; Cat #107621; Lot B24168), F4/80 Pacific Blue (Clone: BM8; Cat # 123123; Lot B217177), Ly-6G PE (Clone: 1A8; Cat # 127607; Lot B235376), Ly-6C (Clone: HK1.4; Cat #128021: Lot B221000), CD11b PE/Cy7 (Clone: M1/70; Cat #101215; Lot B249267). For antigen uptake into draining lymph node immune cells, the following antibodies were obtained from Biolegend: I-A/I-E Pacific Blue (Clone: M5/114.15.2; Cat #107619; Lot: B252426), CD11c APC (Clone: N418; Cat #117310; Lot: B253461), F4/80 PE (Clone: BM8; Cat #123109; Lot: B251636) were used. For GC B cells staining, the following antibodies against GL7 Pacific Blue (Clone: GL7; Cat #144613; Lot: B244647), CD95 PE (Clone: SA367H8; Cat #152607; Lot: B239352), B220 APC (Clone: RA3-6B2; Cat #103211; Lot: B205878) were used. For assessing Tfh cells, the following antibodies were obtained from Biolegend: CXCRS APC (Clone: L138D7 Cat #145505; Lot B243491), PD-1 PE (Clone: 29F.1Al2; Cat #135205; Lot: B251877), Alexa Fluor 488 CD4 (Clone: GK1.5; Cat #100425; Lot: B238433). For intracellular cytokine staining: Surface markers to identified CD4⁺ and CD8⁺ T cells including, TCRβ APC/Cy7 (Clone: H57-597; Cat #109219), CD4 PE/Cy7 (Clone: RM4-4; Cat #116015), CD8 PreCP/Cy5.5 (Clone: 53-5.8; Cat #140417), CD44 BV605 (Clone: IM7; Cat #563058), Live/Dead marker (Cat #L34957); Intracellular markers including IFNγ Pacific Blue (Clone: XMG1.2; Cat #505817), TNFa PE (Clone: MP6-XT22; Cat #506305), Foxp3 Alex Fluor 488 (Clone: ME-14, Cat #126405), IL2 PE/TexasRed.(Clone: JES6-5H4; Cat #503839).

Cell culture: RAW264.7 murine macrophage cells were obtained from ATCC and cultured in in Dulbecco's modified Eagle's medium (DMEM) with 10% fetal bovine serum (FBS) and 1% penicillin/streptomycin (Pen/Strep). HEK293T cells were provided by Bruce Davidson from the University at Buffalo, cells were cultured in DMEM with 10% FBS, 1% Pen/Strep and 10 mM sodium pyruvate. HEK293T-ACE2 cells were provided by Huihui Mu from the Scripps Research Institute, cells were cultured in DMEM with 10% FBS and 1% Pen/Strep, 10 mM sodium pyruvate and 2 μg/ml of puromycin. Bone marrow dendritic cells (BMDC) were derived from naive CD-1 mice and cultured in RPMI medium with 10% FBS, 1% Pen/Strep and 20 ng/ml of GM-CSF. Bone marrow was collected from the femurs and tibia of mice. The concentration of cells was seeded at 10⁷ cells/ml and cultured in 10 cm petri dish in RPMI 1640 culture medium with 10% FBS and 20 ng/ml of recombinant GM-CSF on day 0. On day 3, an additional 10 ml RPMI medium containing GM-CSF was added so the final volume of the medium was 20 ml. On day 6, non-adherent cells were collected and cultured in a 24-well plate at 5×10 ⁵ cell/ml in RPMI culture medium containing 10% FBS and 1% Pen/Strep and then incubated for 24 hr with CoPoP/MPLA, CoPoP/MPLA/QS21, and PBS mixed at 1 μg/mL RBD, with a fixed 4:1 mass ratio of CoPoP to RBD. Cells were washed 3× with PBS containing 0.1% BSA and 0.05% sodium azide, and stained with antibodies against CD11c-APC/Cy7, CD40-Pacific blue, CD80-APC, CD86-APC and MHC-II-PE for 30 min on ice prior to flow cytometry.

Liposome preparation: Liposomes were prepared by an ethanol injection method, followed by nitrogen-pressurized lipid extrusion in phosphate buffered saline (PBS) carried out at 60° C. Remaining ethanol was removed by dialysis against PBS twice at 4° C. For liposomes containing QS21, QS21 (1 mg/ml) was added to the liposomes after formation at an equal mass ratio as MPLA. Final liposome concentration was adjusted to 320 μg/ml CoPoP and were passed through a 0.2 μm sterile filter and stored at 4° C. Liposome sizes and polydispersity index were determined by dynamic light scattering (DLS) with a NanoBrook 90 plus PALS instrument after 200-fold dilution in PBS. The CoPoP/MPLA liposome formulation had a mass ratio of [DPPC: CHOL: MPLA: CoPoP] [4:2:0.4:1], CoPoP/MPLA/QS21 liposome formulation had a mass ratio of [DOPC: CHOL: MPLA: CoPoP: QS21] [20:5:0.4:1:0.4], PoP/MPLA liposomes served as the control liposomes which have a similar formulation as CoPoP/MPLA liposomes but lack of cobalt in the porphyrin-phospholipid, this formulation had a mass ratio of [DPPC: CHOL: MPLA: PoP] [4:2:0.4:1] and AS01-like liposome formation had a mass ratio of [DOPC: CHOL: MPLA: QS21] [20:5:0.4:0.4].

Slot Blot for antigen conformation: Liposomal samples were prepared as follows, CoPoP liposomes (320 μg/ml of CoPoP) mixed with RBD antigens (80 μg/ml) at antigen: CoPoP=1:4 mass ratio, Pfs25 (a malaria antigen) with CoPoP liposomes served as a negative control in this experiment. The 48-well slot blot (Cat #M1706545) was set up as per instructions. The gasket support plate was placed onto the vacuum manifold, then the sealing gasket was put on top of the support plate. The nitrocellulose membrane was pre-wetted in PBS for 10 min at room temperature, then placed on top of the sealing gasket. The 24-well sample template was put on top of the membrane and secured by tightening the screws. Fifty μl of mixed samples were slowly applied into each well, and the entire sample was allowed to flow through the membrane by gravity. The membrane was removed and blocked using 5% BSA in PBS for 30 min at RT, followed by incubating with 1000× diluted human ACE2, Fc Tag (cat #AC2-H5257 from Acrobiosystems) for 1 hr at RT. The membrane was washed with PBS for 5 mins twice, followed by incubation with HRP anti-human IgG (cat #109-035-098 from Jackson ImmunoResearch) for 30 min at RT. After incubation, the membrane was washed for 5 min with PBS 2 times. The membrane was imaged using a Bio-Rad ChemiDoc™ Imager.

Ni-NTA competition binding test: To check RBD antigen binding stability, Ni-NTA Magnetic Beads (ThermoFisher cat #88831) were used to compete with pre-bound proteins to the liposomes (1:4 mass ratio of total protein: CoPoP). Sufficient beads were added to ensure full binding of the free proteins in the sample. The samples were incubated with the beads for 30 min before the supernatant and magnetic beads were separated and collected using a magnetic separator (ThermoFisher cat #12321D). The beads were then resuspended in PBS. Denaturing reducing loading dye was then added to all samples (supernatant and beads) and heated near 100° C. for 10 min. The samples were then loaded into a Novex 4-12% Bis-Tris acrylamide gel (Invitrogen cat #NP0321BOX) and subjected to PAGE and bands were visualized with Coomassie staining.

Fluorophore-labeled RBD: RBD was labeled with DY-490-NHS-Ester (DY-490). Labeling was carried out with DY-490 to RBD at molar ratio of 10:1. 100 μg of RBD was first dialysis against 100 mM sodium bicarbonate buffer (pH 9) for 4-6 hr at 4° C. twice, and then labeled with DY-490 for 1 hr at room temperature with continues stirring. Free dye was removed by dialysis against PBS three times at 4° C.

Fluorescent quenching assay: DY-490-labeled RBD was carried out by incubating antigens and liposomes with a 1:4 mass ratio of RBD: CoPoP or PoP at the final antigen concentration at 40 μg/ml. The quenching of each sample was assessed at 0.5, 1, 2, and 3 hr at RT. To check the fluorescence signal, each of the incubation samples were diluted for 200 times in PBS in a 96-well plate, and the fluorescence signal was measured at excitation/emission of 491/515. The percentage of binding was calculated based on the following formula: % antigen binding=[1-FL_{liposomes+antigen}/FL_{antigen}]×100% (FL=fluorescent intensity).

Serum stability: The mixture of DY-490 labeled RBD (80 μg/ml) with CoPoP liposomes (320 μg/ml CoPoP) were incubated for 3 hr at room temperature followed by adding the same amount of 40% human serum in PBS into the sample to achieve a final concentration at 20% human serum. Samples were incubated at 37° C. for the indicated durations.

Soluble and particle form of RBD binding assay to HEK293T-ACE2 cells: 5×10⁵ cells of HEK293T cells or HEK293T-ACE2 cells were incubated with labeled RBD (0.5 μg/ml) with CoPoP/MPLA liposomes and CoPoP/MPLA/QS21 liposomes and PBS alone for 20 min on ice. After incubation, the cells were washed with ice-cold PBS twice. The cells were lysis with lysis buffer (0.1% triton with 20 μg/ml proteinase K) at 60° C. for min. The samples were placed in a 96-well plate, and fluorescence signal were check at excitation/ emission at 491/515 for DY-490 labeled RBD. Liposome detected on hACE2 coated plate: 1 μg/mL of hACE2 in coating

buffer (3.03g Na₂CO₃; 6 g NaHCO₃ in 1 L distilled water, pH 9.6) were coated on the plate for 2 h at 37° C. Wells were washed and blocked with 2% BSA in PBS for 2 h at 37° C. At the meantime, RBD with CoPoP/MPLA*liposomes and CoPoP/MPLA/QS21*liposomes were incubated for 3 hrs. RBD (0.4 μg/ml of RBD) with CoPoP liposomes were added into each wells and incubated for 1 hr at RT. The wells were wash with PBS for 4 time, and 200 μl of

PBS containing 0.5% Triton X were added into each well to break the liposomes. The CoPoP liposomes in this study contained a small amount of PoP lipid, liposomal formulation is CoPoP/MPLA*liposomes (DPPC: Chol: MPLA: CoPoP: PoP=4:2::0.4:0.8:0.2) and CoPoP/1MPLA/QS21* liposomes (DOPC: Chol: MPLA: QS21: CoPoP: PoP=20:5:0.4:0.4:0.8:0.2).

Pseudovirus production: HEK293T cells were seeded at 5×10⁵ cells/ml in a T75 flask overnight with DMEM medium with 10% FBS. When the cells were approximately 60% confluent they were transfected with the retroviral vector pQCXIX encoding firefly luciferase (FLuc), a plasmid expressing MLV gag and pol proteins, and a plasmid expressing the S protein of SARS-CoV-2 protein at a ratio of 5:5:1 by mass. Eleven μg of total DNA was mixed with 44 μg of PEI at room temperature for 20 min, then the mixture was slowly added to the cells. After 6 hr of incubation at 37° C., the medium was replaced with 10 ml of complete DMEM medium and the culture was incubated at 32° C. After 48 hr post transfection, the cultured medium containing pseudovirus was harvested, and passed through a 0.45 μm pore size filter and the virus supernatant was supplemented with 10 mM HEPES, aliquoted and stored at −80° C.

Cryo-electron microscopy: 20 μl of RBD-HEK293 (80 μg/ml) was mixed with 20 μl of CoPoP/MPLA liposomes or CoPoP/MPLA/QS21 liposomes in PBS. Holey carbon grids (c-flat CF-2/2-2C-T) were washed with chloroform and glow discharged at 5 mA for 15 seconds immediately before the application of the sample. A volume of 3.6 μl of sample was deposited in the grid and vitrification was performed in a Vitrobot (ThermoFisher) by blotting the grids once for 3 seconds and blot force +2 before they were plunged into liquid ethane. Temperature and relative humidity during the vitrification process were maintained at 25° C. and 100%, respectively. Grids were loaded into the Tecnai F20 electron microscope operated at 200 kV using a Gatan 626 single tilt cryo-holder. Images were collected in a Gatan Ultrascan 4000 4k×4k CCD Camera System Model 895 at a nominal magnification 60,000×, which produced images with a calibrated pixel size of 1.8 Å/pixel. Images were collected with a total dose of ˜50 e-/Å 2 using a defocus ranging from −2.7 to −3.5 μm. Images were cropped and prepared for figures using the Adobe Photoshop program.

Murine immunization and serum analysis: 5-week-old female CD-1 mice (ordered from Envigo RMS LLC) received intramuscular injections on days 0 and 14 containing 100 ng RBD combined with the following liposomal adjuvants: CoPoP/MPLA liposomes with the following formulation, [DPPC: CHOL: MPLA: CoPoP] of [4:2:0.4:1], CoPoP/MPLA/QS21 liposomes with the following formulation, [DOPC: CHOL: MPLA: CoPoP:QS21] of [4:2:0.4:1:0.4], PoP/MPLA liposomes with the following formulation, [DPPC: CHOL: MPLA: PoP] of [4:2:0.4:1], AS01-like liposomes with the following formulation, [DOPC: CHOL: MPLA: CoPoP:QS21] of [4:2:1:1:1]. The following adjuvants were used for comparison of the CoPoP liposomal adjuvant versus commercial adjuvants, ISA720, Alum and Addavax.

Splenocytes harvest to check RBD specific cytokines. Splenocytes were harvest from the immunized mice on day 28. Spleen were collected and than passed through a μm cell strainer in a 50 mL tube to collect single cell. Cells were centrifuged at 500 rcf, and red blood lysis buffer were added for 5 min on ice to lysed red blood cells. After incubation, 20 mL of PBS were added to dilute the lysis buffer, and samples were centrifuge at 500 rcf for 5 min. In 96-well culture plate, 2.5×10⁵ cells/well were stimulated with 1 μl/ml of RBD and cultured in RPMI medium, with 10% FBS, 1% Pen/Strep, 1 mM pyruvate and 1 mM non-essential amino acid, 50 μM 2-Mercaptoethanol. In order to check IFNγ secretion, cultured medium was collected after 48 hr, and IFNγ secretion level were measured based on IFNγ mouse ELISA kit (fisher Scientific, Cat. 50-183-06). On the other side, in order to check cytokines in specific T cells, splenocytes were stimulated with 1 μl/ml of RBD for 18 h, followed by incubation with brefeldin A (Biosciences, Cat. #555029) for another 6 h to block the cytokine secretion from the cells. Cells were stained for the surface markers using TCRβ APC/Cy7, CD4 PE/Cy7, CD8 PreCP/Cy5.5, CD44 BV605, Live/Dead marker (Cat. L34957) diluted in FASC buffer (cold-PBS containing 0.5% BSA and 0.05% sodium azide) for 25 min on ice. The cells were washed with FASC buffer twice, then fixed with the fixation/permeabilization buffer (BD cytofix/perm kit; Biosciences Cat. #555028) for 10 min on ice. The cells were wash twice with FASC buffer, and permeabilization buffer (BD cytofix/perm kit; Biosciences Cat. #555028) were added into each wells for 20 min on ice. Intracellular markers including IFNγ Pacific Blue, TNFα PE, Foxp3 Alex Fluor 488, IL2 PE/TexasRed were diluted in permeabilization buffer, and cells were stained for 25 min on ice. Stained cells were washed twice with permeabilization buffer, then resuspended in FASC buffer prior to BD LSRFortessa TM X-20 flow cytometry.

New Zealand white rabbit immunization: 10-12 weeks old female rabbits received intramuscular injections on days 0 and 21 of 20 μg of RBD-HEK293 with CoPoP/MPLA liposomes with a [DPPC: CHOL: MPLA: CoPoP=4:2:0.4:1] mass ratio or with CoPoP/MPLA/QS21 liposomes with a [DPPC: CHOL: MPLA: CoPoP: QS21=20:5:0.4:1:0.4] mass ratio. Serum was collected on day 0, 21 and day 42.

ELISA assay: Anti-RBD IgG titer was assessed by ELISA in 96-well plates. 2.5 μg/ml of RBD in coating buffer (3.03 g Na₂CO₃; 6 g NaHCO₃ in 1 L distilled water, pH 9.6) were coated on the plate for 2 h at 37° C. Wells were washed and blocked with 2% BSA in PBS containing 0.1% Tween-20 (PBS-T) for 2 h at 37° C. Mouse sera (diluted in PBS-T containing 1% BSA) were incubated in the wells, followed by washing with PBS-T. Goat anti-mouse IgG-HRP was added. Wells were washed again with PBS-T before addition of tetramethylbenzidine solution. Titers were defined as the reciprocal serum dilution at which the absorbance at 450 nm exceeded background by greater than 0.5 absorbance units.

Pseudovirus based neutralization assay: HEK293T-ACE2 cells were seeded into 96 well plate at a density of 2×10⁵ cells/well for overnight. Immunized sera from mice and rabbit with serial dilution were incubated with pseudovirus at room temperature for 30 min, then 50 μl of pseudovirus with sera at different dilutions were added to each well after removing 50 μl of cultured medium, and the cells were cultured for 48 hr. The medium was removed from each well and the cells were washed with 200 μL PBS, followed by adding 30 μl of lysis buffer (Promega E1500) for 10 min. The lysate was transferred into a white plate, and 100 μl of substrate were added. CentroPRO (Cat. #LB 962) was used to measure the luciferase activity.

RBD-hACE2 inhibition assay: SARS-CoV-2 Surrogate Virus Neutralization Test (sVNT) Kit (GenScript, Cat. L00847) were used to check if post immune sera could bock the interaction between hACE2 and HRP-RBD antigen. Mice sera was diluted 100× and rabbit sera was diluted 20× with sample dilution buffer. Positive and negative controls were included in the kit, and the control vials were diluted 10×. The diluted positive and negative controls, as well as the diluted samples were mixed with HRP-RBD solution at a 1:1 volume, then incubated at 37° C. for 30 min. 100 μl of these mixtures were loaded into the wells of an ELISA plate pre-coated with hACE2 and incubated at 37° C. for 15 min. The plate was washed 4× to remove unbound HRP-RBD. Percentage of Inhibition=(1−OD₄₅₀ post immune sera/OD₄₅₀ negative control)×100.

VNT assay (Live virus neutralization test): The ability of plasma samples to neutralize SARS-CoV-2 host-cell infection was determined with a traditional VN assay using a SARS-CoV-2 isolate deposited by the Centers for Disease Control and Prevention and obtained through BEI Resources, NIAID, NIH: SARS-Related Coronavirus 2, Isolate USA-WA1/2020, NR-52281. The assay was performed in triplicate, and a series of eight two-fold serial dilutions of the serum was assessed. One-hundred tissue culture infective dose 50 (TCID₅₀) units of SARS-CoV-2 was added to two-fold dilutions of serum and incubated for 1 hr at 37° C. The virus and serum mixture were added to Vero E6 cells grown in a 96-well microtiter plates, incubated for 3 d, after which the host cells were treated for 1 h with crystal violet-formaldehyde stain (0.013% crystal violet, 2.5% ethanol, and 10% formaldehyde in 0.01 M PBS). The endpoint of the microneutralization assay was designated as the highest plasma dilution at which all three or two of three wells were not protected from virus infection, as assessed by visual examination.

Lymph node studies for RBD uptake: Mice were immunized with 1 μof RBD-DY490 with CoPoP/MPLA, CoPoP/MPLA/QS21, Alum or AS01-like liposome. After 48 hr, mice were sacrificed and inguinal lymph nodes were collected. Lymph node were pass through a 70 μm cell strainer and 5×10⁵ cells per tube were stained with the following murine antibodies against I-A/I-E, B220, CD11c or F4/80 (all from BioLegend) for 30 min at room temperature. The samples were washed with FASC buffer twice prior to BD LSRFortessa TM X-20 flow cytometry. Flowjo (version 10) software was used for data analysis. GC cells and Tfh cell populations: Mice received 100 ng of RBD adjuvanted

with CoPoP/MPLA, CoPoP/MPLA/QS21 or Alum. 7 days after immunization, mice were sacrificed and the inguinal LN were collected. Lymph nodes were pass through a 70 μm cell strainer and 5×10⁵ cells per tube were than stained with antibodies against B220, CD95, GL7. CD4, CXCRS or PD-1 for 30 min on ice. The samples were washed with FASC buffer twice prior to BD LSRFortessa TM X-20 flow cytometry. Flowjo (version 10) software was used for data analysis.

For lymph node cell recruitment: Mice were injected intramuscularly with CoPoP/PHAD liposomes or Alum with 100 ng of Pfs25. 48 hr after injection, mice were sacrifice and lymph nodes were collected for cell extraction. Cells were stained with combination antibodies against Ly6C, CD11b, Ly6G, CD11c, CD3, I-A/I-E and F4/80, for 30 min on ice. The samples were washed with FASC buffer twice prior to BD LSRFortessa TM X-20 flow cytometry. Flowjo (version 10) software was used for data analysis. Cells were first gated with CD11c and CD11b, then immune cells were identified based on surface marker in CD11c^{high} and CD11b^{low}, neutrophils (Ly6G^{high}), eosinophils (Ly6G^{int}, F4/80^{int}, SSC), monocytes (Ly6C^{high}) and macrophage (F4/80^{high}). Three types of DC cells were gated, for myeloid DC, we first gate Cd11c^{high} and CD11b^{high}, then gated MHC-II positive cells.

Acute toxicity studies: 8-week-old female CD-1 mice were treated with intramuscular injection of CoPoP/MPLA/RBD or CoPoP/MPLA/QS21/RBD with 1 μg RBD. Two weeks later, blood and serum were collected and subjected to standard complete blood cell count and serum panel (IDEXX Cat #98-20590-00).

Local reactogenicity of adjuvants: Mice received 1 μg of RBD admixed with different types of adjuvants were injected into the footpads, and 50 μL sample per mouse were used. For CoPoP/MPLA liposomes, CoPoP/MPLA/QS21 liposomes, AS01 liposomes, 1:1 mass ratio of RBD (80 μg/mL) to liposomes were incubated for 3 h at room temperature. For Alum and Addavax, 1:1 mass ratio of RBD (80 μg/mL) to adjuvant were admixed directly before injection. Montanide ISA720 were mixed with PBS and vortexed at maximum speed for 40 minutes at 3:7 volume ratio of Montanide ISA720: PBS. The mice received 50 μL adjuvant samples into their left footpad and 50 μL of PBS into their right footpad as a control. Thickness of the footpad was measured by caliper 48 hrs after footpad injection and swelling was calculated by the following formula: [Thickness_{left} footpad−Thickness_{right} footpad].

While the invention has been described through specific embodiments, routine modifications will be apparent to those skilled in the art and such modifications are intended to be within the scope of the present disclosure.

What is claimed is:

1. A method for generating neutralizing antibodies against SARS-CoV-2 virus in subject comprising administering to the subject a vaccine composition comprising:
a) liposomes comprising i) a bilayer, wherein the bilayer comprises phospholipid, and porphyrin having cobalt coordinated thereto forming cobalt-porphyrin; and a polyhistidine-tagged amino acid sequence of receptor-binding-domain (RBD) of Spike protein from SARS-CoV-2, wherein at least a portion of the polyhistidine tag resides in the hydrophobic portion of the bilayer and one or more histidines of the polyhistidine tag are coordinated to the cobalt in the cobalt-porphyrin, and wherein at least a portion of the amino acid sequence is exposed to the outside of the liposome; and
b) a pharmaceutical carrier.

2. The method of claim 1, wherein the RBD has a sequence depicted as SEQ ID NO:1 or a variant thereof which has at least 90% identity with the sequence of SEQ ID NO:l.

3. The method of claim 1, wherein the cobalt porphyrin is conjugated to a phospholipid to form a cobalt porphyrin-phospholipid conjugate.

4. The method of claim 1, wherein the cobalt porphyrin-phospholipid conjugate makes up from 0.1 to 25 mol % of the bilayer.

5. The method of claim 1, wherein the cobalt porphyrin-phospholipid conjugate makes up from 5 to 10 mol % of the bilayer.

6. The method of claim 1, wherein the bilayer further comprises cholesterol.

7. The method of claim 1, wherein the polyhistidine-tag comprises 6 to 10 histidine residues.

8. The method of claim 1, wherein size of the liposome is 50 nm to 200 nm.

9. The method of claim 1, wherein the liposomes further comprise one or more adjuvants.

10. The method of claim 1, wherein the one or more adjuvants are attenuated lipid A derivatives, phosphorylated hexaacyl disaccharides, and/or QS21.

11. The method of claim 1, further comprising one or more adjuvants which are not associated with the liposomes.

12. The method of claim 1, wherein the subject is a human.

13. The method of claim 1, wherein the composition is administered multiple times.

14. A method of generating an immune response of asubject against SARS-CoV-2 comprising:
a) providing a receptor binding domain (RBD) of Spike protein of SARS-CoV-2, the RBD further comprising a polyhistidine tag;
b) complexing the polyhistidine tagged RBD with liposomes comprising i) a bilayer, wherein the bilayer comprises cobalt-porphyrin-phospholipids such that at least a portion of the polyhistidine tag is incorporated into the hydrophobic portion of the bilayer one or more histidines of the polyhistidine tag are coordinated to the cobalt in the cobalt-porphyrin-phospholipid, at least a portion of the RBD is exposed to the outside of the liposome; and
c) administering the complexed RBD to the subject thereby generating an immune response,
wherein the immune response of the subject against SARS-CoV-2 is greater than the immune response generated by administration of the RBD in the absence of complexing the RBD with the liposomes.

15. The method of claim 14 wherein the RBD has a sequence set forth in SEQ ID NO:1 or a sequence that is at least 90% homologous to the sequence set forth in SEQ ID NO:1.

16. The method of claim 15, wherein the immune response is humoral.

17. The method of claim 15, wherein the immune response is cellular.

18. The method of claim 15, wherein the immune response is both humoral and cellular.

19. The method of claim 17 or 18, wherein the cellular immune response comprises an increase in CD4+ or CD8+ T cells.

20. The method of claim 14, wherein the RBD has a sequence depicted as SEQ ID NO:1 or a variant thereof which has at least 90% identity with the sequence of SEQ ID NO:1.

21. The method of claim 14, wherein the cobalt porphyrin is conjugated to a phospholipid to form a cobalt porphyrin-phospholipid conjugate.

22. The method of claim 14, wherein the cobalt porphyrin-phospholipid conjugate makes up from 0.1 to 25 mol % of the bilayer.

23. The method of claim 14, wherein the cobalt porphyrin-phospholipid conjugate makes up from 5 to 10 mol % of the bilayer.

24. The method of claim 14, wherein the bilayer further comprises cholesterol.

25. The method of claim 14, wherein the polyhistidine-tag comprises 6 to 10 histidine residues.

26. The method of claim 14, wherein size of the liposome is 50 nm to 200 nm.

27. The method of claim 14, wherein the liposomes further comprise one or more adjuvants.

28. The method of claim 14, wherein the one or more adjuvants are attenuated lipid A derivatives, phosphorylated hexaacyl disaccharides, and/or QS21.

29. The method of claim 14, further comprising one or more adjuvants which are not associated with the liposomes.

30. The method of claim 14, wherein the subject is a human.

31. The method of claim 14, wherein the composition is administered multiple times.

32. A vaccine composition comprising
a) liposomes comprising i) a bilayer, wherein the bilayer comprises phospholipid, and porphyrin having cobalt coordinated thereto forming cobalt-porphyrin; and ii) a polyhistidine-tagged amino acid sequence of receptor-binding-domain (RBD) of Spike protein from SARS-CoV-2, wherein at least a portion of the polyhistidine tag resides in the hydrophobic portion of the bilayer and one or more histidines of the polyhistidine tag are coordinated to the cobalt in the cobalt-porphyrin, and wherein at least a portion of the amino acid sequence is exposed to the outside of the liposome; and
b) a pharmaceutical carrier.

33. The vaccine composition of claim 32, wherein the RBD has a sequence set forth in SEQ ID NO:1 or a variant thereof which has at least 90% identity with the sequence of SEQ ID NO:1.

34. The vaccine composition of claim 32, wherein the cobalt porphyrin-phospholipid conjugate makes up from 0.1 to 25 mol % of the bilayer.

35. The vaccine composition of claim 34, wherein the cobalt porphyrin-phospholipid conjugate makes up from 5 to 10 mol % of the bilayer.

36. The vaccine composition of claim 32, wherein the polyhistidine-tag comprises 6 to 10 histidine residues.

37. The vaccine composition of claim 32, wherein size of the liposome is 50 nm to 200 nm.

38. The vaccine composition of claim 32, wherein the liposomes further comprise one or more adjuvants.

39. The vaccine composition of claim 38, wherein the one or more adjuvants are attenuated lipid A derivatives, phosphorylated hexaacyl disaccharides, and/or QS21.

40. The vaccine composition of claim 32, further comprising one or more adjuvants which are not associated with the liposomes.
